(19) United States
(12) Patent Application Publication
(10) Pub. No.: US 2011/0129921 A1
(43) Pub. Date: Jun. 2, 2011

(54) TARGETED POLYMER BIOCONJUGATES

(75) Inventors: Paul H. Johnson, Snohomish, WA (US); Patrick S. Stayton, Seattle, WA (US); Allan S. Hoffman, Seattle, WA (US); Anthony J. Convertine, Seattle, WA (US); Craig L. Duvall, Nashville, TN (US); Danielle Benoit, Rochester, NY (US); Chen-Chang Lee, Oceanside, CA (US); Robert W. Overell, Shoreline, WA (US); Anna S. Gall, Woodinville, WA (US); Mary G. Prieve, Lake Forest Park, WA (US); Amber E.E. Paschal, Redmond, WA (US); Charbel Diab, Seattle, WA (US); Priyadarsi De, Mohanpur (IN)

(73) Assignees: UNIVERSITY OF WASHINGTON, Seattle, WA (US); PHASERX, INC., Seattle, WA (US)

(21) Appl. No.: 12/992,545

(22) PCT Filed: May 13, 2009
(86) PCT No.: PCT/US09/43839

§ 371 Date: Feb. 10, 2011

Related U.S. Application Data
(60) Provisional application No. 61/171,365, filed on Apr. 21, 2009.
(60) Provisional application No. 61/091,294, filed on Aug. 22, 2008.
(60) Provisional application No. 61/120,769, filed on Dec. 8, 2008.
(60) Provisional application No. 61/112,054, filed on Nov. 6, 2008.
(60) Provisional application No. 61/140,779, filed on Dec. 24, 2008.
(60) Provisional application No. 61/052,914, filed on May 13, 2008.
(60) Provisional application No. 61/052,908, filed on May 13, 2008.
(60) Provisional application No. 61/112,048, filed on Nov. 6, 2008.
(60) Provisional application No. 61/140,774, filed on Dec. 24, 2008.

(51) Int. Cl. C12N 5/071; C08F 8/40; B32B 9/02

(52) U.S. Cl. 435/375; 525/54.2; 525/54.1; 428/402

(57) ABSTRACT

Polymer bioconjugate having a RNAi agent covalently coupled to the alpha or omega end of a pH-dependent membrane-destabilizing polymer.

### CROSS-REFERENCE

This application claims the benefit of U.S. Provisional Application No. 61/052,908, filed May 13, 2008, U.S. Provisional Application No. 61/052,914, filed May 13, 2008, U.S. Provisional Application No. 61/091,294 filed Aug. 22, 2008, U.S. Provisional Application No. 61/112,048 filed Nov. 6, 2008, U.S. Provisional Application No. 61/140,774 filed Dec. 24, 2008, U.S. Provisional Application No. 61/112,054 filed Nov. 6, 2008, U.S. Provisional Application No. 61/140,779 filed Dec. 24, 2008, U.S. Provisional Application No. 61/120,769 filed Dec. 8, 2008, U.S. Provisional Application No. 61/171,365 filed Apr. 21, 2009, each of which is hereby incorporated by reference in its entirety.

### STATEMENT AS TO FEDERALLY SPONSORED RESEARCH

This invention was made with Government support under Contract No. NIH1RO1EB002991, awarded by the National Institutes of Health. The Government has certain rights in the invention.

### FIELD OF THE INVENTION

Described herein are bioconjugates formed from polymers and the use of polymer bioconjugates.

### BACKGROUND OF THE INVENTION

In certain instances, it is beneficial to introduce therapeutic or diagnostic or biomolecular agents (e.g., oligonucleotides) into living cells. In some instances, delivery of such polynucleotides to a living cell provides a therapeutic benefit.

### SUMMARY OF THE INVENTION

In some embodiments, provided herein are copolymer bioconjugates comprising at least one monomeric unit that is hydrophilic (e.g., at physiologic pH), and one or more hydrophobic moiety. In certain embodiments, a polymer bioconjugate provided herein comprises a membrane-destabilising polymer (i.e., is destabilizing of a cellular membrane), such as, by way of non-limiting example, an extracellular membrane, an intracellular membrane, a vesicle, an organelle, an endosome, a liposome, or a red blood cell. Preferably, in certain instances, wherein a polymer bioconjugate described herein is in contact with a cellular membrane, it destabilizes the membrane and enters the intracellular environment.

Provided in certain embodiments herein is a polymer bioconjugate, the polymer bioconjugate comprising:
a. a pH-dependent membrane-destabilizing polymer having an alpha end and an omega end, and comprising
   i. a plurality of monomeric units comprising a species that is anionic at about neutral pH, and
   ii. a plurality of monomeric units comprising a hydrophobic group, and
b. a RNAi agent covalently coupled to the alpha end or the omega end of the membrane-destabilizing polymer.

In specific embodiments, the pH-dependent membrane-destabilizing polymer is a block copolymer.

Provided in some embodiments herein is a polymer bioconjugate comprising:
a. a pH-dependent membrane-destabilizing polymer having an alpha end and an omega end, the pH-dependent membrane destabilizing polymer comprising:
   i. a plurality of monomeric units comprising a species that is anionic at about neutral pH; and
   ii. a plurality of monomeric units comprising a hydrophobic group; and
b. an RNAi agent covalently coupled to the alpha end or the omega end of the membrane-destabilizing polymer.

In some specific embodiments, the polymer bioconjugate does not stay associated as a micelle when diluted below a concentration of 100 μg/mL or 50 ug/mL in water at a neutral pH. In certain specific embodiments, any polymer bioconjugate has a particle size of not more than 30 nm. In specific embodiments, any polymer bioconjugate provided herein has a particle size (diameter) of not more than 20 nm, 25 nm, 20 nm, 15 nm, 10 nm, or 5 nm.

Provided in certain embodiment herein is a polymer bioconjugate comprising
a. a hydrophilic pH-dependent membrane-destabilizing polymer having an alpha end and an omega end, and comprising;
   i. a plurality of monomeric units comprising a species that is anionic at about neutral pH, and
   ii. a plurality of monomeric units comprising a hydrophobic group, and
b. a RNAi agent covalently coupled to the alpha end or the omega end of the membrane-destabilizing polymer.

Provided in some embodiments herein is a polymer bioconjugate comprising
a. a pH-dependent membrane-destabilizing polymer having an alpha end and an omega end, and consisting essentially of a random copolymer, the random copolymer comprising
   i. a plurality of monomeric units comprising a species that is anionic at about neutral pH, and
   ii. a plurality of monomeric units comprising a hydrophobic group, and
b. a RNAi agent covalently coupled to the alpha end or the omega end of the membrane-destabilizing polymer.

Provided in some embodiments herein is a polymer bioconjugate comprising:
a. a pH-dependent membrane-destabilizing polymer having an alpha end and an omega end, the pH-dependent membrane destabilizing polymer comprising:
   i. a plurality of monomeric units comprising a species that is anionic at about neutral pH; and
   ii. a plurality of monomeric units comprising a hydrophobic group; and
b. an RNAi agent covalently coupled to the alpha end or the omega end of the membrane-destabilizing polymer; the polymer bioconjugate having a particle size of not more than 30 nm. In specific embodiments, any polymer bioconjugate provided herein has a particle size (diameter) of not more than 20 nm, 25 nm, 20 nm, 15 nm, 10 nm, or 5 nm.

In certain embodiments, the RNAi agent of any polymer bioconjugate described herein is a polynucleotide. In specific embodiments, the polynucleotide is an siRNA. In some embodiments, the polynucleotide is a dicer substrate. In some embodiments, the polynucleotide comprises 5′ and a 3′ end, and wherein the polynucleotide is coupled to the membrane-destabilizing polymer at either the 5′ or 3′ end of the polynucleotide.

In certain embodiments, the RNAi agent of any polymer bioconjugate described herein is covalently coupled to the membrane-destabilizing polymer through a linking moiety. In some embodiments, the linking moiety comprises at least one bond which is a cleavable bond. In certain embodiments, the linking moiety is a non-cleavable linking moiety. In some embodiments, the linking moiety is cleavable and comprises a dicer substrate. In certain embodiments, the linking moiety is cleavable and comprises a disulfide. In some embodiments, the RNAi agent is coupled to the pH-dependent membrane-destabilizing polymer through a plurality of linking moieties.

In certain embodiments, the anionic species of any of the polymer bioconjugates described herein are substantially anionic at about neutral pH. In some embodiments, at least 50%, at least 60%, at least 70%, at least 80%, at least 90% or at least 95% of such species are anionic at about neutral pH. That is, near the pKₐ of such a species, there exists an equilibrium between anionic and non-anionic species. Thus, if the aforementioned species has a pKₐ at or around neutral pH, an equilibrium population of such species will be anionic and a population will be non-anionic.

In certain embodiments, any polymer bioconjugate described herein further comprises one or more targeting ligand covalently coupled or attached to the pH-dependent membrane-destabilizing polymer. In some embodiments, the one or more targeting ligands are selected from the group consisting of a vitamin, a saccharide, a peptide, a hormone, an aptamer, a small molecule, or combinations thereof. In some embodiments, the one or more targeting ligand is or comprises a peptide. In specific embodiments, the peptide is an antibody or an antibody fragment. In some embodiments, the one or more targeting ligands are coupled to the membrane-destabilizing polymer at the alpha end or at the omega end opposing the RNAi agent, and/or to a pendant group of one or more monomeric units of the membrane-destabilizing polymer. In certain embodiments, any polymer bioconjugate provided herein comprises a targeting ligand that is covalently coupled to the end of the membrane-destabilizing polymer opposing the RNAi agent. In some embodiments, any polymer bioconjugate described herein comprises a targeting ligand that is coupled to the membrane-destabilizing polymer through a linking moiety. In some embodiments, the linking moiety coupling the targeting ligand to the membrane-destabilizing polymer is a cleavable linking moiety.

In certain embodiments, provided herein is a compound comprising a plurality of polymer bioconjugates, each polymer bioconjugate independently being any bioconjugate described herein and comprising a membrane-destabilizing polymer coupled to the same targeting ligand, and coupled to the RNAi agent. In specific embodiments, each membrane-destabilizing polymer is coupled to the RNAi agent at either the alpha or omega end of the polymer, and is coupled to the targeting ligand at the opposing end of the polymer. In some embodiments, the compound comprises greater than 2, greater than 5, or greater than 10 RNAi agents coupled to the targeting ligand.

In some embodiments, provided herein is any polymer bioconjugate described herein with the RNAi agent coupled to the alpha end of the membrane-destabilizing polymer. In specific embodiments, such a polymer bioconjugate further comprises a targeting ligand coupled to the omega end and/or a pendant group of a monomeric unit of the membrane-destabilizing polymer. In certain embodiments, provided herein is any polymer bioconjugate described herein with the RNAi agent coupled to the omega end of the membrane-destabilizing polymer. In specific embodiments, such a polymer bioconjugate further comprises a targeting ligand coupled to the alpha end or to a pendant group of a monomeric unit of the membrane-destabilizing polymer.

In some embodiments, any polymer bioconjugate described herein further comprises one or more charge shielding moieties (e.g., polyethylene glycol (PEG) moiety), e.g., as or on pendant groups of the monomeric units of the membrane destabilizing polymer. In some embodiments, the membrane-destabilizing polymer is substituted or functionalized with a charge shielding group (e.g., PEG), e.g., substituted on a pendant group of the membrane-destabilizing polymer.

In certain embodiments, any polymer bioconjugate described herein comprises a membrane-destabilizing polymer that is a copolymer comprising a plurality of different monomeric units. In specific embodiments, the copolymer is a random copolymer.

In certain embodiments, any polymer bioconjugate described herein comprises a pH-dependent membrane-destabilizing polymer comprising a plurality of hydrophilic units. In certain embodiments, any polymer bioconjugate described herein comprises a pH-dependent membrane-destabilizing polymer comprising a plurality of hydrophilic units wherein one or more of the plurality of hydrophilic monomeric units comprises a hydroxyalkyl, polyoxyalkyl or alkoxyalkyl moiety. In certain embodiments, the one or more of the hydrophilic monomeric units comprise a polyethylene glycol (PEG) moiety. In some embodiments, the membrane-destabilizing polymer comprises x monomeric units comprising the chargeable species being chargeable to an anion and y hydrophobic monomeric units comprising the hydrophobic groups, wherein the ratio of x to y is about 1:1 to about 5:1. In some embodiments, any polymer bioconjugate described herein comprises a pH dependent membrane destabilizing polymer is comprises a plurality of monomeric units comprising both a chargeable species and a hydrophobic group. In some embodiments, the pH dependent membrane destabilizing polymer comprises more than 5, more than 10, more than 20, more than 50 or more than 100 chargeable species that are chargeable to anions. In some embodiments, the pH dependent membrane destabilizing polymer comprises more than 5, more than 10, more than 20, more than 50 or more than 100 hydrophobic groups or species.

In some embodiments, each chargeable species being chargeable to an anion is independently, by way of non-limiting example, a carboxylic acid, anhydride, sulfonamide, sulfonic acid, sulfinic acid, sulfuric acid, phosphoric acid, phosphinic acid, phosphonic acid, boric acid, or phosphorous acid. In specific embodiments, at least one of monomeric units comprising a chargeable species being chargeable to an anion of a membrane destabilizing polymer provided in a polymer bioconjugate described herein is alkyl acrylic acid.

In certain embodiments, hydrophobic monomeric units utilized herein include, by way of non-limiting example, alkyl acrylate or alkyl alkacrylate.

In some embodiments, any polymer bioconjugate provided herein comprises a membrane-destabilizing polymer that comprises a monomeric unit comprising a conjugatable group, e.g., a means functional group, or a reactive functional group (masked or non-masked). In some embodiments, a monomeric unit comprising a conjugatable group is a click monomeric unit. In certain embodiments, a monomeric unit comprising the conjugatable group is at the omega end of the membrane-destabilizing polymer and is covalently conjugated to the RNAi agent. In some embodiments, a conjugatable group is conjugated to the RNAi agent through a linking moiety.

In certain embodiments, provided herein is a polymer bioconjugate comprising a membrane-destabilizing polymer that is produced by living polymerization, living radical polymerization, or a combination thereof. In some embodiments, the membrane-destabilizing polymer is produced by living radical polymerization. In specific embodiments, the membrane-destabilizing polymer is produced by reversible addition-fragmentation chain transfer (RAFT) polymerization. In some embodiments, the RAFT polymerization comprises polymerizing the membrane-destabilizing polymer in the presence of a chain transfer agent (CTA) that comprises a modifiable group. In some embodiments, the modifiable group of the chain transfer agent is masked or non-masked.

In some embodiments, a polymer bioconjugate provided herein comprises a pH dependent membrane destabilizing polymer further comprising an additional chargeable species, wherein each additional chargeable species is chargeable to a cation.

Provided in certain embodiments herein is a compound comprising
a. a polymer bioconjugate comprising
   i. a pH-dependent membrane-destabilizing polymer having an alpha end and an omega end, the pH-dependent membrane destabilizing polymer comprising:
      1. a plurality of monomeric units comprising a chargeable species, each chargeable species being anionic at about neutral pH, and
      2. a plurality of monomeric units comprising hydrophobic groups, and
   ii. an RNAi agent covalently coupled to the alpha end or the omega end of the membrane-destabilizing polymer; and
b. a proteinaceous targeting ligand covalently coupled to the pH-dependent membrane-destabilizing polymer.

In some embodiments, the compound comprises a plurality of polymer bioconjugates covalently coupled to the proteinaceous targeting ligand. In certain embodiments, the compound comprises at least 2, at least 5, or at least 10 polymer bioconjugates covalently coupled to the proteinaceous tageting ligand. In some embodiments, the proteinaceous targeting ligand is covalently coupled to the pH dependent membrane destabilizing polymer at the end opposite the RNAi agent. In certain embodiments, the proteinaceous tageting ligand is covalently coupled to the pH dependent membrane destabilizing polymer at a pendant group of a monomeric unit of the pH dependent membrane destabilizing polymer.

### INCORPORATION BY REFERENCE

All publications and patent applications mentioned in this specification are herein incorporated by reference for the purposes cited to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 describes the polymer design for Poly[HPMA]-b-[(PAA)ₓ(BMA)_{y}(DMAEMA)_{z}]ₘ.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### DISCUSSION

Provided in certain embodiments herein are polymer bioconjugates and processes for making the same. In some embodiments, a polymer bioconjugate provided herein comprises an RNAi agent coupled to an alpha end or an omega end of a pH-dependent membrane destabilizing polymer. In some embodiments, the polymer bioconjugate, or pH-dependent membrane destabilizing polymer thereof, comprises a plurality of monomeric units having chargeable species that are chargeable to an anion, and a plurality of monomeric units having hydrophobic groups. In some embodiments, the polymer bioconjugates also comprise one or more targeting ligands. In specific embodiments, the polymer comprises ethylenic backbone. In some embodiments, the polymer bioconjugates do not self-assemble into supramolecular assemblies, e.g. micelles. In certain embodiments, the polymer bioconjugates are sub-30 nm particles. In certain embodiments, the polymer bioconjugates are sub-30 nm, sub-25 nm, sub-20 nm, sub-15 nm, sub-10 nm, or sub-5 nm particles.

In some instances, provided herein are polymer bioconjugates for the delivery of RNAi agents (including, e.g oligonucleotides) to a living cell. In certain embodiments, the polymer bioconjugates provided herein are biocompatible, non-toxic (e.g., exhibit low toxicity), and/or reproducibly synthesized. Additionally, in some embodiments, the polymer bioconjugates protect the RNAi agent (e.g., oligonucleotide) payload from degradation, enter living cells via a naturally occurring process (e.g., by endocytosis), and/or deliver the RNAi agent (e.g., oligonucleotide) into the cytoplasm of a living cell after being contacted with the cell. In certain instances, the RNAi agent (e.g., oligonucleotide) is an siRNA and/or another ‘nucleotide-based’ agent that alters the expression of at least one gene in the cell. Accordingly, in certain embodiments, the polymer bioconjugates provided herein are useful for delivering siRNA into a cell. In certain instances, the cell is in vitro, and in other instances, the cell is in vivo. In some embodiments, a therapeutically effective amount of the polymer bioconjugate comprising an siRNA is administered to an individual in need thereof (e.g., in need of having a gene knocked down, wherein the gene is capable of being knocked down by the siRNA administered). In specific instances, the polymer bioconjugates are useful for or are specifically designed for delivery of siRNA to specifically targeted cells of the individual.

### DEFINITIONS

It is understood that, with regard to this application, use of the singular includes the plural and vice versa unless expressly stated to be otherwise. That is, “a” and “the” refer to one or more of whatever the word modifies. For example, “the polymer” or “a nucleotide” may refer to one polymer or nucleotide or to a plurality of polymers or nucleotides. By the same token, “polymers” and “nucleotides” would refer to one polymer or one nucleotide as well as to a plurality of polymers or nucleotides unless, again, it is expressly stated or obvious from the context that such is not intended.

As used herein, two moieties or compounds are “attached” if they are held together by any interaction including, by way of non-limiting example, one or more covalent bonds, one or more non-covalent interactions (e.g., ionic bonds, static forces, van der Waals interactions, combinations thereof, or the like), or a combination thereof.

Aliphatic or aliphatic group: the term “aliphatic” or “aliphatic group”, as used herein, means a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms.

Aryl or aryl group: as used herein, the term “aryl” or “aryl group” refers to monocyclic, bicyclic, and tricyclic ring systems having a total of five to fourteen ring members, wherein at least one ring in the system is aromatic and wherein each ring in the system contains three to seven ring members.

As used herein, a “charge neutralized” means a particle having a Zeta potential that is between ±10 to ±30 mV, and/or the presence of a first number (z) of chargeable species that are chargeable to a negative charge (e.g., acidic species that become anionic upon de-protonation) and a second number (0.5·z) of chargeable species that are chargeable to a positive charge (e.g., basic species that become cationic upon protonation).

Heteroalkyl: the term “heteroalkyl” means an alkyl group wherein at least one of the backbone carbon atoms is replaced with a heteroatom.

Heteroaryl: the term “heteroaryl” means an aryl group wherein at least one of the ring members is a heteroatom.

As used herein, a “chargeable species”, “chargeable group”, or “chargeable monomeric unit” is a species, group or monomeric unit in either a charged or non-charged state. In certain instances, a “chargeable monomeric unit” is one that can be converted to a charged state (either an anionic or cationic charged state) by the addition or removal of an electrophile (e.g., a proton (H⁺), for example in a pH dependent manner). The use of any of the terms “chargeable species”, “chargeable group”, or “chargeable monomeric unit” includes the disclosure of any other of a “chargeable species”, “chargeable group”, or “chargeable monomeric unit” unless otherwise stated. A “chargeable species” that is “charged or chargeable to an anion” or “charged or chargeable to an anionic species” is a species or group that is either in an anionic charged state or non-charged state, but in the non-charged state is capable of being converted to an anionic charged state, e.g., by the removal of an electrophile, such as a proton (H+). In specific embodiments, a chargeable species is a species that is charged to an anion at about neutral pH. It should be emphasized that not every chargeable species on a polymer will be anionic at a pH near the pKₐ (acid dissociation constant) of the chargeable species, but rather an equilibrium of anionic and non-anionic species will co-exist. A “chargeable species” that is “charged or chargeable to a cation” or “charged or chargeable to a cationic species” is a species or group that is either in an cationic charged state or non-charged state, but in the non-charged state is capable of being converted to a cationic charged state, e.g., by the addition of an electrophile, such as a proton (H+). In specific embodiments, a chargeable species is a species that is charged to an cation at about neutral pH. It should be emphasized that not every charged cationic species on a polymer will be cationic at a pH near the pKₐ (acid dissociation constant) of the charged cationic species, but rather an equilibrium of cationic and non-cationic species will co-exist. “Chargeable monomeric units” described herein are used interchangeably with “chargeable monomeric residues”. The phrase “chargeable species being chargeable to an anion” (or the like) at a particular pH and the phrase “a species that is anionic” (or the like) at a particular pH are used synonymously and interchangeably herein.

Heteroatom: the term “heteroatom” means an atom other than hydrogen or carbon, such as oxygen, sulfur, nitrogen, phosphorus, boron, arsenic, selenium or silicon atom.

Hydrophobic species: “hydrophobic species” (used interchangeably herein with “hydrophobicity-enhancing moiety”), as used herein, is a moiety such as a substituent, residue or a group which, when covalently attached to a molecule, such as a monomer or a polymer, increases the molecule's hydrophobicity or serves as a hydrophobicity enhancing moiety. The term “hydrophobicity” is a term of art describing a physical property of a compound measured by the free energy of transfer of the compound between a non-polar solvent and water (Hydrophobicity regained. Karplus P.A., Protein Sci., 1997, 6: 1302-1307.) A compound's hydrophobicity can be measured by its logP value, the logarithm of a partition coefficient (P), which is defined as the ratio of concentrations of a compound in the two phases of a mixture of two immiscible solvents, e.g. octanol and water. Experimental methods of determination of hydrophobicity as well as methods of computer-assisted calculation of logP values are known to those skilled in the art. Hydrophobic species of the present invention include but are not limited to aliphatic, heteroaliphatic, aryl, and heteroaryl groups.

Inhibition: The terms “inhibition,” “silencing,” and “attenuation” as used herein refer to a measurable reduction in expression of a target mRNA or the corresponding protein as compared with the expression of the target mRNA or the corresponding protein in the absence of a knockdown agent. “Knockdown”, or the reduction in expression of the target mRNA or the corresponding protein, can be assessed by measuring the mRNA levels using techniques well known in the art such as quantitative polymerase chain reaction (qPCR) amplification, RNA solution hybridization, nuclease protection, northern blotting and hybridization, and gene expression monitoring with a microarray; and in the case of proteins by techniques well known in the art such as SDS-PAGE, antibody binding, western blot analysis, immunoprecipitation, radioimmunoassay or enzyme-linked immunosorbent assay (ELISA), fluorescence activated cell analysis and immunocytochemistry.

As used herein, a “linking moiety” or a “linker” is a chemical bond or a multifunctional (e.g., bifunctional) residue which is used to link an RNAi agent, e.g., an oligonucleotide, and/or a targeting agent to the pH-dependent membrane destabilizing polymer. Linker moieties comprise any of a variety of compounds which can form an amide, ester, ether, thioether, carbamate, urea, amine or other linkage, e.g., linkages which are commonly used for immobilization of biomolecules in affinity chromatography. In some embodiments, the linking moiety comprises a cleavable bond, e.g. a bond that is unstable and/or is cleaved upon changes in certain intracellular parameters (e.g., pH or redox potential). In some embodiments, the linking moiety is non-cleavable. In certain embodiments, the linking moiety is attached to the RNAi agent or a targeting agent by one or more covalent bonds. In some embodiments, the linking moiety is attached to the pH-dependent membrane destabilizing polymer through one or more covalent bonds.

Without being bound by theory not expressly recited in the claims, a membrane destabilizing polymer can directly or indirectly elicit a change (e.g., a permeability change) in a cellular membrane structure (e.g., an endosomal membrane) so as to permit an agent (e.g., polynucleotide), in association with or independent of a polymer, to pass through such membrane structure—for example to enter a cell or to exit a cellular vesicle (e.g., an endosome). A membrane destabilizing polymer can be (but is not necessarily) a membrane disruptive polymer. A membrane disruptive polymer can directly or indirectly elicit lysis of a cellular vesicle or disruption of a cellular membrane (e.g., as observed for a substantial fraction of a population of cellular membranes).

Generally, membrane destabilizing or membrane disruptive properties of polymers or micelles can be assessed by various means. In one non-limiting approach, a change in a cellular membrane structure can be observed by assessment in assays that measure (directly or indirectly) release of an agent (e.g., polynucleotide) from cellular membranes (e.g., endosomal membranes)—for example, by determining the presence or absence of such agent, or an activity of such agent, in an environment external to such membrane. Another non-limiting approach involves measuring red blood cell lysis (hemolysis)—e.g., as a surrogate assay for a cellular membrane of interest. As used herein, a “polymer bioconjugate” is a covalent conjugate of at least one polymer and at least one therapeutic agent (e.g., RNAi agent), diagnostic agent, or research reagent.

As used herein, a “micelle” includes a particle comprising a core and a hydrophilic shell, wherein the core is held together at least partially, predominantly or substantially through hydrophobic interactions. In certain instances, as used herein, a “micelle” is a multi-component, nanoparticle comprising at least two domains, the inner domain or core, and the outer domain or shell. The core is at least partially, predominantly or substantially held together by hydrophobic interactions, and is present in the center of the micelle. As used herein, the “shell of a micelle” is defined as non-core portion of the micelle.

A “pH dependent membrane-destabilizing polymer” is a polymer that undergoes a transition from at least partially, predominantly, or substantially hydrophilic to at least partially, predominantly, or substantially hydrophobic in a pH-dependent manner and is membrane destabilizing in a pH-dependent manner. In certain instances, a pH dependent membrane destabilizing polymer comprises a plurality of hydrophobic species and a plurality of chargeable species that are chargeable to an anion. In some embodiments, the chargeable species is anionic at about neutral pH. In further or alternative embodiments, the chargeable species is non-charged at a lower pH, e.g., endosomal pH. In some embodiments, the pH-dependent membrane destabilizing polymer comprises a plurality of chargeable species that are chargeable to a cation and/or chargeable monomeric units, which comprise chargeable species that are chargeable to cations. In some embodiments, the pH dependent membrane-destabilizing polymer comprises non-peptidic and/or non-lipidic polymer backbone.

As used herein, the terms neutral pH, physiologic and physiological pH are synonymous and interchangeable. The terms do not include the acidic pH found in endosomes, or the like.

Oligonucleotide gene expression modulator: as used herein, an “oligonucleotide gene expression modulator” is an oligonucleotide agent capable of inducing a selective modulation (e.g., increase or decrease) of gene expression in a living cell by mechanisms including but not limited to an antisense mechanism or by way of an RNA interference (RNAi)-mediated pathway which may include (i) transcription inactivation; (ii) mRNA degradation or sequestration; (iii) transcriptional inhibition or attenuation or (iv) inhibition or attenuation of translation. Oligonucleotide gene expression modulators include virtually any regulatory RNA, such as but not limited to antisense oligonucleotides, miRNA, siRNA, RNAi, shRNA, aptamers and any analogs or precursors thereof.

Oligonucleotide knockdown agent: as used herein, an “oligonucleotide knockdown agent” is an oligonucleotide species which inhibits gene expression by targeting and binding an intracellular nucleic acid in a sequence-specific manner. Non-limiting examples of oligonucleotide knockdown agents include siRNA, miRNA, shRNA, dicer substrates, antisense oligonucleotides, decoy DNA or RNA, antigene oligonucleotides and any analogs and precursors thereof.

As used herein, the term “oligonucleotide” refers to a polymer comprising 7-200 nucleotide monomeric units. In some embodiments, “oligonucleotide” encompasses single and or/double stranded RNA as well as single and/or double-stranded DNA. Furthermore, the terms “nucleotide”, “nucleic acid,” “DNA,” “RNA,” and/or similar terms include nucleic acid analogs, i.e. analogs having a modified backbone, including but not limited to peptide nucleic acids (PNA), locked nucleic acids (LNA), phosphono-PNA, morpholino nucleic acids, or nucleic acids with modified phosphate groups (e.g., phosphorothioates, phosphonates, 5′-N-phosphoramidite linkages). Nucleotides can be purified from natural sources, produced using recombinant expression systems and optionally purified, chemically synthesized, etc. As used herein, a “nucleoside” is the term describing a compound comprising a monosaccharide and a base. The monosaccharide includes but is not limited to pentose and hexose monosaccharides. The monosaccharide also includes monosaccharide mimetics and monosaccharides modified by substituting hydroxyl groups with halogens, methoxy, hydrogen or amino groups, or by esterification of additional hydroxyl groups. In some embodiments, a nucleotide is or comprises a natural nucleoside phosphate (e.g. adenosine, thymidine, guanosine, cytidine, uridine, deoxyadenosine, deoxythymidine, deoxyguanosine, and deoxycytidine phosphate). In some embodiments, the base includes any bases occurring naturally in various nucleic acids as well as other modifications which mimic or resemble such naturally occurring bases. Nonlimiting examples of modified or derivatized bases include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid, wybutoxosine, pseudouracil, queosine, 2-thiocytosine,

5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil, 2-aminoadenine, pyrrolopyrimidine, and 2,6-diaminopurine. Nucleoside bases also include universal nucleobases such as difluorotolyl, nitroindolyl, nitropyrrolyl, or nitroimidazolyl. Nucleotides also include nucleotides which harbor a label or contain abasic, i.e. lacking a base, monomers. A nucleic acid sequence is presented in the 5′ to 3′ direction unless otherwise indicated. A nucleotide can bind to another nucleotide in a sequence-specific manner through hydrogen bonding via Watson-Crick base pairs. Such base pairs are said to be complementary to one another. An oligonucleotide can be single stranded, double-stranded or triple-stranded.

RNA interference (RNAi): As used herein, the term “RNA interference” or “RNAi” refers to sequence-specific inhibition of gene expression and/or reduction in target mRNA and protein levels mediated by an at least partially double-stranded RNA, which also comprises a portion that is substantially complementary to a target RNA.

RNAi agent: As used herein, the term “RNAi agent” refers to an agent, e.g., an oligonucleotide, which can mediate inhibition of gene expression through an RNAi mechanism, including but not limited to siRNA, microRNA (miRNA), short hairpin RNA (shRNA), dicer substrates and the precursors thereof.

Short interfering RNA (siRNA): As used herein, the term “short interfering RNA” or “siRNA” refers to an RNAi agent comprising a nucleotide duplex that is approximately 15-50 base pairs in length and optionally further comprises zero to two single-stranded overhangs. One strand of the siRNA includes a portion that hybridizes with a target RNA in a complementary manner. In some embodiments, one or more mismatches between the siRNA and the targeted portion of the target RNA may exist. In some embodiments, siRNAs mediate inhibition of gene expression by causing degradation of target transcripts.

Short hairpin RNA (shRNA): Short hairpin RNA (shRNA) refers to an oligonucleotide having at least two complementary portions hybridized or capable of hybridizing with each other to form a double-stranded (duplex) structure and at least one single-stranded portion.

Inhibit gene expression: As used herein, the phrase “inhibit gene expression” means to cause any measurable reduction in the amount of an expression product of the gene. An expression product includes an RNA transcribed from the gene (e.g. an mRNA) and/or a polypeptide translated from an mRNA transcribed from the gene. The level of expression may be determined using standard techniques for measuring mRNA or protein.

Dicer Substrate: a “dicer substrate” is a greater than approximately 25 base pair duplex RNA that is a substrate for the RNase III family member Dicer in cells. Dicer substrates are cleaved to produce approximately 21 base pair duplex small interfering RNAs (siRNAs) that evoke an RNA interference effect resulting in gene silencing by mRNA knockdown.

As used herein, “substantially non-charged” includes a Zeta potential potential that is between ±10 to ±30 mV, and/or the presence of a first number (z) of chargeable species that are chargeable to a negative charge (e.g., acidic species that become anionic upon de-protonation) and a second number (0.5·z) of chargeable species that are chargeable to a positive charge (e.g., basic species that become cationic upon protonation).

Therapeutic agent: As used herein, the phrase “therapeutic agent” refers to any agent that, when administered to a subject, organ, tissue, or cell has a therapeutic effect and/or elicits a desired biological and/or pharmacological effect.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition.

pH-Dependent Membrane Destabilizing Polymers

In some embodiments, pH dependent membrane destabilizing polymers provided herein are membrane destabilizing at any suitable pH. In some embodiments, the pH dependent membrane destabilizing polymers are membrane destabilizing (e.g., in an aqueous medium) at an endosomal pH, a pH of about 6.5, or lower, about 5.0 to about 6.5, or about 6.2, or lower.

In specific embodiments, the pH dependent membrane destabilizing polymers provided herein comprise a plurality of anionic chargeable groups, species, or monomeric units and a plurality of hydrophobic species, groups, or monomeric units. In more specific embodiments, the chargeable groups, species, or monomeric units are chargeable to anionic groups, species, or monomeric units. In various embodiments, each chargeable group, species, or monomeric unit within a pH dependent membrane destabilizing polymer is independently in a charged or non-charged state.

In certain embodiments, the pH dependent membrane destabilizing polymers destabilize an endosomal membrane in a pH-dependent manner. In some embodiments, at or near physiological pH (e.g., pH of circulating blood), the polymers are minimally membrane-destabilizing (e.g., are substantially non-membrane destabilizing), but upon exposure to decreased pH (e.g., endosomal pH), the polymers are membrane-destabilizing. In certain instances, this transition to a membrane-destabilizing state occurs via the protonation of the negatively charged chargeable species that are incorporated into the polymers, such protonation leading to an increase in the hydrophobicity of the polymers. In certain instances, the increased hydrophobicity of the polymer results in a conformational change of the polymer. In some instances, the conformation change of the polymer provides a membrane destabilizing polymer and/or membrane destabilizing polymer bioconjugate.

In certain embodiments, polymer bioconjugates described herein comprise pH dependent membrane destabilizing polymers, wherein the polymers are non-peptidic and/or non-lipidic. In some embodiments, the backbone of the pH dependent membrane destabilizing polymers forming the polymer bioconjugate is non-peptidic and/or non-lipidic. As used herein, lipids are a diverse group of compounds broadly defined as hydrophobic or amphiphilic molecules that originate entirely or in part from two distinct types of biochemical subunits: ketoacyl and isoprene groups, e.g., fatty acids, glycerolipids, glycerophoispholipids, sphingolipids, saccharolipids, polyketides, sterol lipids, and prenol lipids.

In specific embodiments, the pH dependent membrane destabilizing polymers are substantially hydrophilic and/or are substantially non-hydrophobic. In more specific embodiments, the pH dependent membrane destabilizing polymers are substantially hydrophilic and/or are substantially non-hydrophobic at about neutral pH and/or at about physiological pH. In some embodiments, the polymers do not comprise a substantially hydrophobic segment and/or a hydrophobic block. In some embodiments, the polymers do not comprise a segment or block that is substantially hydrophobic at about physiologic pH. In various instances, a “polymeric segment” is a polymer section with a given physical property (e.g., a physical property of a block described herein, e.g., hydrophobicity, hydrophilicity, chargeability, etc.) or which comprises one or more blocks with similar physical properties (e.g., hydrophobicity, hydrophilicity, chargeability, etc.).

In certain embodiments, a polymer bioconjugate provided herein comprises a plurality of chargeable monomeric units comprising a chargeable species, the chargeable species being chargeable to an anion In some embodiments, the chargeable monomeric units are chargeable to anionic species upon removal of a proton (H⁺). In specific embodiments, the chargeable monomeric units with chargeable species that are chargeable to anions are non-charged or substantially neutral at an acidic pH (e.g., an endosomal pH, a pH below about 6.5, a pH below about 6.0, a pH below about 5.8, a pH below about 5.7, a pH below about 5.6, a pH below about 5.5, a pH below about 5.0, a pH below about 4.5 or the like). In specific embodiments, the pKa of the chargeable monomeric units with chargeable species that are chargeable to anions are, independently, about 4.5 to about 7, about 4.5 to about 6.5, about 4.5 to about 6, about 4.5 to about 5.8, or any other suitable pKa. In certain embodiments, the chargeable monomeric units comprise chargeable species that are anionic at an approximately physiological pH. In some embodiments, the chargeable species is chargeable to an anionic species upon deprotonation. In some embodiments, the pH dependent membrane destabilizing polymer comprises more than 5, more than 10, more than 20, more than 50 or more than 100 chargeable species that are chargeable to anions.

In some embodiments, a polymer bioconjugate provided herein comprises a plurality of hydrophobic monomeric units. In certain embodiments, the hydrophobic monomeric units comprise hydrophobic species. In some embodiments, the pH dependent membrane destabilizing polymer comprises more than 5, more than 10, more than 20, more than 50 or more than 100 hydrophobic groups or species In some embodiments, the hydrophobic species are present on the anionic chargeable monomeric units. In some embodiments, the ratio of the hydrophobic monomeric units to the monomeric units comprising a chargeable species that is chargeable to an anion is between about 1:6 and about 1:1, about 1:5 and about 1:1, about 1:4 and about 1:1, about 1:3 and about 1:1, about 1:2 and about 1:1 at about a neutral pH.

In some embodiments, a polymer bioconjugate provided herein comprises a plurality of hydrophilic monomeric units. In specific embodiments, the hydrophilic monomeric units are substantially non-chargeable, e.g., meaning that the hydrophilic monomeric units are substantially non-charged at physiological pH (e.g., pH about neutral such as 7.2-7.4). In certain embodiments, the hydrophilic monomeric units comprise hydrophilic groups (e.g., hydroxyl groups, thiol groups, PEG groups or other polyoxylated alkyl groups, or the like, or a combination thereof). In some embodiments, the pH dependent membrane destabilizing polymer comprises more than 5, more than 10, more than 20, more than 50 or more than 100 hydrophilic groups or species.

In some embodiments, the pH dependent membrane destabilizing polymers are copolymers. In specific embodiments, the pH dependent membrane destabilizing copolymer is a monoblock polymer or a multiblock polymer (e.g., a diblock polymer). The term “copolymer”, as used herein, signifies that the polymer is the result of polymerization of two or more different monomers. A “monoblock polymer” is a synthetic product of a single polymerization step. The term monoblock polymer includes a copolymer (i.e. a product of polymerization of more than one type of monomers) and a homopolymer (i.e. a product of polymerization of a single type of monomers). A “block” copolymer refers to a structure comprising one or more sub-combination of constitutional or monomeric units. In some embodiments, the block copolymer is a diblock copolymer. A diblock copolymer comprises two blocks; a schematic generalization of such a polymer is represented by the following: [AₐB_{b}C_{c} . . . ]ₘ−[XₓY_{y}Z_{z} . . . ]ₙ, wherein each letter stands for a constitutional or monomeric unit, and wherein each subscript to a constitutional unit represents the mole fraction of that unit in the particular block, the three dots indicate that there may be more (there may also be fewer) constitutional units in each block and m and n indicate the molecular weight of each block in the diblock copolymer. As suggested by the schematic, in some instances, the number and the nature of each constitutional unit is separately controlled for each block. The schematic is not meant and should not be construed to infer any relationship whatsoever between the number of constitutional units or the number of different types of constitutional units in each of the blocks. Nor is the schematic meant to describe any particular number or arrangement of the constitutional units within a particular block. In each block the constitutional units may be disposed in a purely random, an alternating random, a regular alternating, a regular block or a random block configuration unless expressly stated to be otherwise. A purely random configuration, for example, may have the non-limiting form: x-x-y-z-x-y-y-z-y-z-z-z . . . . A non-limiting, exemplary alternating random configuration may have the non-limiting form: x-y-x-z-y-x-y-z-y-x-z . . . , and an exemplary regular alternating configuration may have the non-limiting form: x-y-z-x-y-z-x-y-z . . . . An exemplary regular block configuration may have the following non-limiting configuration: . . . x-x-x-y-y-y-z-z-z-x-x-x . . . , while an exemplary random block configuration may have the non-limiting configuration: . . . x-x-x-z-z-x-x-y-y-y-y-z-z-z-x-x-z-z-z- . . . . In a gradient polymer, the content of one or more monomeric units increases or decreases in a gradient manner from the a end of the polymer to the ω end. In none of the preceding generic examples is the particular juxtaposition of individual constitutional units or blocks or the number of constitutional units in a block or the number of blocks meant nor should they be construed as in any manner bearing on or limiting the actual structure of block copolymers forming the polymer bioconjugate of this invention.

As used herein, the brackets enclosing the constitutional units are not meant and are not to be construed to mean that the constitutional units themselves form blocks. That is, the constitutional units within the square brackets may combine in any manner with the other constitutional units within the block, i.e., purely random, alternating random, regular alternating, regular block or random block configurations. The copolymers described herein are, optionally, alternate, gradient or random copolymers. In some instances, the pH-dependent membrane-destabilizing polymer consists essentially of a random copolymer.

In some embodiments, the polymer bioconjugates provided herein do not associate (e.g., self-assemble) into structured nanoparticles (e.g., micelles). In some embodiments, the polymer bioconjugates do not self-associate through hydrophobic interactions. In specific embodiments, the polymer bioconjugates do not self-assemble through interactions that are substantially, or predominantly hydrophobic. In certain embodiments, the polymer bioconjugates do not rapidly self-assemble into a micelle when the polymer bioconjugate is at a concentration of 1-10 mg/mL, less than 0.1 mg/mL, less than 1 mg/mL, less than 5 mg/mL, or less than 10 mg/mL in aqueous solution (e.g., water, saline, plasma and the like) at a neutral pH. In certain embodiments, the polymer bioconjugate does not rapidly self-assemble into a micelle when the polymer bioconjugate is at a concentration of 1-10 g/mL, less than 0.1 mg/mL, less than 1 mg/mL, less than 5 mg/mL, or less than mg/mL in aqueous solution (e.g., water, saline, plasma and the like) at a neutral pH at an approximately room temperature or at approximately 37° C. In certain embodiments, the polymer bioconjugate does not self-assemble into a micelle in 5 min, in 10 min, in 20 min, in 30 min, in 1 hr, in 6 hrs. In some embodiments, any polymer bioconjugate provided herein has a particle size of not more than about 30 nm, not more than about 25 nm, not more than about 20 nm, not more than about nm, not more than about 10 nm, not more than about 5 nm or not more than 1 nm.

In certain embodiments, a polymer bioconjugate provided herein comprises a pH dependent membrane destabilizing polymer, an RNAi agent, and a polypeptide targeting agent (e.g., an antibody). In specific embodiments, such a polymer bioconjugate has a particle size of not more than about 100 nm, not more than about 90 nm, not more than about 80 nm, not more than about 70 nm, not more than about 60 nm, not more than about 50 nm, not more than about 40 nm, not more than about 30 nm, not more than about 20 nm, or not more than about 10 nm. In some embodiments, provided herein is a compound comprising a polypeptide targeting agent (e.g., antibody) conjugated to a plurality of polymer bioconjugates, wherein the polymer bioconjugates each independently comprise a pH dependent membrane destabilizing polymer, and an RNAi agent. In specific embodiments, such a compound has a particle size of not more than about 100 nm, not more than about 90 nm, not more than about 80 nm, not more than about 70 nm, not more than about 60 nm, not more than about 50 nm, not more than about 40 nm, not more than about 30 nm, not more than about 20 nm, or not more than about 10 nm.

In certain embodiments, copolymers (e.g., pH dependent membrane destabilizing copolymers) of the polymeric carriers provided herein comprise ethylenically unsaturated monomers. The term “ethylenically unsaturated monomer” is defined herein as a compound having at least one carbon double or triple bond. The non-limiting examples of the ethylenically unsaturated monomers are: an alkyl (alkyl)acrylate, a methacrylate, an acrylate, an alkylacrylamide, a methacrylamide, an acrylamide, a styrene, an allylamine, an allylammonium, a diallylamine, a diallylammonium, an N-vinyl formamide, a vinyl ether, a vinyl sulfonate, an acrylic acid, a sulfobetaine, a carboxybetaine, a phosphobetaine, or maleic anhydride.

In various embodiments, any monomer suitable for providing the polymers (e.g., the pH dependent membrane destabilizing polymers) of the polymer bioconjugates described herein is used. In some embodiments, monomers suitable for use in the preparation of the polymers (e.g., the membrane destabilizing block polymers) of the polymer bioconjugates provided herein include, by way of non-limiting example, one or more of the following monomers: methyl methacrylate, ethyl acrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate (DMAEMA), N,N-diethylaminoethyl methacrylate, triethyleneglycol methacrylate, oligoethyleneglycol methacrylate, oligoethyleneglycol acrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolacrylamide, N-ethylolacrylamide, vinyl benzoic acid (all isomers), diethylaminostyrene (all isomers), alpha-methylvinyl benzoic acid (all isomers), diethylamino alpha-methylstyrene (all isomers), p-vinylbenzenesulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropylmethacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysillpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-arylmaleimide, N-phenylmaleimide, N-alkylmaleimide, N-butylimaleimide, N-vinylpyrrolidone, N-vinylcarbazole, butadiene, isoprene, chloroprene, ethylene, propylene, 1,5-hexadienes, 1,4-hexadienes, 1,3-butadienes, 1,4-pentadienes, vinylalcohol, vinylamine, N-alkylvinylamine, allylamine, N-alkylallylamine, diallylamine, N-alkyldiallylamine, alkylenimine, acrylic acids, alkylacrylates, acrylamides, methacrylic acids, alkylmethacrylates, methacrylamides, N-alkylacrylamides, N-alkylmethacrylamides, styrene, vinylnaphthalene, vinyl pyridine, ethylvinylbenzene, aminostyrene, vinylpyridine, vinylimidazole, vinylbiphenyl, vinylanisole, vinylimidazolyl, vinylpyridinyl, vinylpolyethyleneglycol, dimethylaminomethylstyrene, trimethylammonium ethyl methacrylate, trimethylammonium ethyl acrylate, dimethylamino propylacrylamide, trimethylammonium ethylacrylate, trimethylanunonium ethyl methacrylate, trimethylammonium propyl acrylamide, dodecyl acrylate, octadecyl acrylate, or octadecyl methacrylate monomers, or combinations thereof.

In some embodiments, functionalized versions of these monomers are optionally used. A functionalized monomer, as used herein, is a monomer comprising a masked or non-masked functional group, e.g. a group to which other moieties can be attached following the polymerization. The non-limiting examples of such groups are primary amino groups, carboxyls, thiols, hydroxyls, azides, and cyano groups. Several suitable masking groups are available (see, e.g., T. W. Greene & P. G. M. Wuts, Protective Groups in Organic Synthesis (2nd edition) J. Wiley & Sons, 1991. P. J. Kocienski, Protecting Groups, Georg Thieme Verlag, 1994, which are incorporated by reference for such disclosure).

Polymers described here are prepared in any suitable manner. Suitable synthetic methods used to produce the polymers provided herein include, by way of non-limiting example, cationic, anionic and free radical polymerization. In some instances, when a cationic process is used, the monomer is treated with a catalyst to initiate the polymerization. Optionally, one or more monomers are used to form a copolymer. In some embodiments, such a catalyst is an initiator, including, e.g., protonic acids (Bronsted acid) or Lewis acids, in the case of using Lewis acid some promoter such as water or alcohols are also optionally used. In some embodiments, the catalyst is, by way of non-limiting example, hydrogen iodide, perchloric acid, sulfuric acid, phosphoric acid, hydrogen fluoride, chlorosulfonic acid, methansulfonic acid, trifluoromehtanesulfonic acid, aluminum trichloride, alkyl aluminum chlorides, boron trifluoride complexes, tin tetrachloride, antimony pentachloride, zinc chloride, titanium tetrachloride, phosphorous pentachloride, phosphorus oxychloride, or chromium oxychloride. In certain embodiments, polymer synthesis is performed neat or in any suitable solvent. Suitable solvents include, but are not limited to, pentane, hexane, dichloromethane, chloroform, or dimethyl formamide (DMF). In certain embodiments, the polymer synthesis is performed at any suitable reaction temperature, including, e.g., from about −50° C. to about 100° C., or from about 0° C. to about 70° C.

In certain embodiments, the polymers are prepared by the means of a free radical polymerization. When a free radical polymerization process is used, (i) the monomer, (ii) optionally, the co-monomer, and (iii) an optional source of free radicals are provided to trigger a free radical polymerization process. In some embodiments, the source of free radicals is optional because some monomers may self-initiate upon heating at high temperature. In certain instances, after forming the polymerization mixture, the mixture is subjected to polymerization conditions. Polymerization conditions are those conditions that cause at least one monomer to form at least one polymer, as discussed herein. Such conditions are optionally varied to any suitable level and include, by way of non-limiting example, temperature, pressure, atmosphere, ratios of starting components used in the polymerization mixture and reaction time. The polymerization is carried out in any suitable manner, including, e.g., in solution, dispersion, suspension, emulsion or bulk.

In some embodiments, initiators are present in the reaction mixture. Any suitable initiator is optionally utilized if useful in the polymerization processes described herein. Such initiators include, by way of non-limiting example, one or more of alkyl peroxides, substituted alkyl peroxides, aryl peroxides, substituted aryl peroxides, acyl peroxides, alkyl hydroperoxides, substituted alkyl hydroperoxides, aryl hydroperoxides, substituted aryl hydroperoxides, heteroalkyl peroxides, substituted heteroalkyl peroxides, heteroalkyl hydroperoxides, substituted heteroalkyl hydroperoxides, heteroaryl peroxides, substituted heteroaryl peroxides, heteroaryl hydroperoxides, substituted heteroaryl hydroperoxides, alkyl peresters, substituted alkyl peresters, aryl peresters, substituted aryl peresters, or azo compounds. In specific embodiments, benzoylperoxide (BPO) and/or AIBN are used as initiators.

In some embodiments, polymerization processes are carried out in a living mode, in any suitable manner, such as but not limited to Atom Transfer Radical Polymerization (ATRP), nitroxide-mediated living free radical polymerization (NMP), ring-opening polymerization (ROP), degenerative transfer (DT), or Reversible Addition Fragmentation Transfer (RAFT). Using conventional and/or living/controlled polymerizations methods, various polymer architectures can be produced, such as but not limited to block, graft, star and gradient copolymers, whereby the monomer units are either distributed statistically or in a gradient fashion across the chain or homopolymerized in block sequence or pendant grafts. In other embodiments, polymers are synthesized by Macromolecular design via reversible addition-fragmentation chain transfer of Xanthates (MADIX) (“Direct Synthesis of Double Hydrophilic Statistical Di- and Triblock Copolymers Comprised of Acrylamide and Acrylic Acid Units via the MADIX Process”, Daniel Taton, et al., Macromolecular Rapid Communications, 22, No. 18, 1497-1503 (2001).)

In certain embodiments, Reversible Addition-Fragmentation chain Transfer or RAFT is used in synthesizing ethylenic backbone polymers of this invention. RAFT is a living polymerization process. RAFT comprises a free radical degenerative chain transfer process. In some embodiments, RAFT procedures for preparing a polymer described herein employs thiocarbonylthio compounds such as, without limitation, dithioesters, dithiocarbamates, trithiocarbonates and xanthates to mediate polymerization by a reversible chain transfer mechanism. In certain instances, reaction of a polymeric radical with the C═S group of any of the preceding compounds leads to the formation of stabilized radical intermediates. Typically, these stabilized radical intermediates do not undergo the termination reactions typical of standard radical polymerization but, rather, reintroduce a radical capable of re-initiation or propagation with monomer, reforming the C═S bond in the process. In most instances, this cycle of addition to the C═S bond followed by fragmentation of the ensuing radical continues until all monomer has been consumed or the reaction is quenched. Generally, the low concentration of active radicals at any particular time limits normal termination reactions.

In some embodiments, pH dependent membrane destabilizing polymers utilized in the polymer bioconjugates provided herein have a low polydispersity index (PDI) or differences in chain length. Polydispersity index (PDI) is determined in any suitable manner, e.g., by dividing the weight average molecular weight of the polymer chains by their number average molecular weight. The number average molecule weight is sum of individual chain molecular weights divided by the number of chains. The weight average molecular weight is proportional to the square of the molecular weight divided by the number of molecules of that molecular weight. Since the weight average molecular weight is always greater than the number average molecular weight, polydispersity is always greater than or equal to one. As the numbers come closer and closer to being the same, i.e., as the polydispersity approaches a value of one, the polymer becomes closer to being monodisperse in which every chain has exactly the same number of constitutional units. Polydispersity values approaching one are achievable using living radical polymerization. Methods of determining polydispersity, such as, but not limited to, size exclusion chromatography, dynamic light scattering, matrix-assisted laser desorption/ionization chromatography and electrospray mass chromatography are well known in the art. In some embodiments, pH dependent membrane destabilizing polymers (e.g., membrane destabilizing copolymers) of the polymer bioconjugates provided herein have a polydispersity index (PDI) of less than 2.0, or less than 1.5, or less than 1.4, or less than 1.3, or less than 1.2.

Polymerization processes described herein optionally occur in any suitable solvent or mixture thereof. Suitable solvents include water, alcohol (e.g., methanol, ethanol, n-propanol, isopropanol, butanol), tetrahydrofuran (THF) dimethyl sulfoxide (DMSO), dimethylformamide (DMF), acetone, acetonitrile, hexamethylphosphoramide, acetic acid, formic acid, hexane, cyclohexane, benzene, toluene, dioxane, methylene chloride, ether (e.g., diethyl ether), chloroform, and ethyl acetate. In one aspect, the solvent includes water, and mixtures of water and water-miscible organic solvents such as DMF.

In some embodiments, a conjugatable group is introduced at the α end of the polymer provided herein by preparing the polymer in the presence of a chain transfer reagent comprising a conjugatable group (e.g., an azide or a pyridyl disulfide group) wherein the conjugatable group is compatible with the conditions of the polymerization process. A non-limiting example of such chain transfer reagent is described by Heredia, K. L et al (see Chem. Commun., 2008, 28, 3245-3247, which is incorporated by reference for the disclosure). In some embodiments, the chain transfer reagent comprises a masked conjugatable group which, following an unmasking reaction, is linked to a siRNA agent or a targeting agent. In some embodiments, a targeting agent, such as but not limited to a small molecule targeting agent (e.g., biotin residue or monosaccharide), is attached at the a end of the polymer provided herein by preparing the polymer in the presence of chain transfer reagent wherein the chain transfer reagent comprises the targeting agent.

In some embodiments, preparation of polymer chains that are selectively functionalized at the ω-terminal end thereof is achieved by ω-terminal chain extension of polymer chains by radical polymerization, such as reversible addition-fragmentation chain transfer (RAFT) polymerization with one or more chain extension residues such as a non-homopolymerizable monomer comprising a conjugatable group (e.g., N-aminoalkylmaleimide) in a manner wherein no more than 10, no more than 5, or no more than 1 monomeric units derived from such monomer are introduced into the ω-terminus of the polymer during the polymerization. ω-Substituted pH dependent membrane destabilizing polymers described herein are prepared in any suitable manner, e.g., a manner set forth in co-pending patent application No. 61/120,756, filed Dec. 8, 2008, which application is incorporated herein by reference for such syntheses.

In certain embodiments, polymeric entities suitable for preparation of the polymer bioconjugates disclosed herein (e.g., copolymers of butyl methacrylate (BMA) and propylacrylic acid (PAA) or copolymers of butyl acrylate (BA) and PAA) are prepared in any suitable manner. In one embodiment, copolymer of BA and PAA is prepared by polymerizing BA and PAA in the presence of the RAFT CTA, ECT, and a radical initiator. In other specific embodiments, the CTA used for co-polymerization of BA and PAA comprises a conjugatable group (e.g. pyridyl disulfide group), thus such polymerization process resulting in a BA/PAA copolymer comprising a conjugatable group at the α terminus of the copolymer. In other embodiments, a conjugatable group is incorporated into the ω end of the copolymer by subjecting the resulting BA/PAA macroCTA to a second RAFT polymerization step involving a non-homopolymerizable monomer. Alternate approaches to obtaining a copolymer with a conjugatable ω end group include reducing the BA/PAA macroCTA to form a thiol end and then covalently attaching a pre-formed linker moiety comprising a different conjugatable group (e.g., a protected amino group or an activated ester of a carboxylic acid) to the formed thiol.

In some instances, the polymers (e.g., pH dependent membrane destabilizing copolymers) comprise conjugatable monomers (e.g., monomers bearing conjugatable groups) which is used for post-polymerization introduction of additional functionalities (e.g. small molecule targeting agents) via know in the art chemistries, for example, “click” chemistry (for example of “click” reactions, see Wu, P.; Fokin, V. V. Catalytic Azide-Alkyne Cycloaddition: Reactivity and Applications. Aldrichim. Acta, 2007, 40, 7-17, which is incorporated by reference). In some embodiments, a monomer comprising such conjugatable groups is co-polymerized with a hydrophobic monomer and a monomer comprising a chargeable to anion species. In some instances, N-hydroxysuccinimide ester of acrylic or alkylacrylic acid is copolymerized with other monomers to form a copolymer which is reacted with amino-functionalized molecules, e.g. targeting ligands or amino derivatives of PEGs. In some embodiments, the monomer comprising a conjugatable group is a pyridyldisulfide acrylate (PDSA).

In some embodiments, the chargeable species, groups, or monomeric units that are chargeable to anionic species, groups, or monomeric units present in the pH-dependent membrane destabilizing polymer are species, groups, or monomeric units that are at least 50%, at least 60%, at least 70%, at least 80%, at least 85%, or at least 95% negatively charged at about neutral pH (e.g., at a pH of about 7.4). In specific embodiments, these chargeable species, groups, or monomeric units are charged by loss of an H⁺, to an anionic species at about neutral pH. In further or alternative embodiments, the chargeable species, groups, or monomeric units that are chargeable to anionic species, groups, or monomeric units present in the polymer are species, groups, or monomeric units that are at least 20%, at least 30%, at least 40%, at least 50%, at least 60%, at least 70%, at least 80%, at least 85%, or at least 95% neutral or non-charged at a slightly acidic pH (e.g., a pH of about 6.5, or less; about 6.2, or less; about 6, or less; about 5.9, or less; about 5.8, or less; about 5.7, or less; about 5.6, or less, about 5.5, or less, about 5.0, or less; or about endosomal pH).

In some embodiments, each chargeable species or group that is chargeable to anionic species or groups is independently, by way of non-limiting example, a carboxylic acid, anhydride, sulfonamide, sulfonic acid, sulfinic acid, sulfuric acid, phosphoric acid, phosphinic acid, boronic acid, phosphorous acid, or the like. Non-limiting examples of anionic chargeable groups also include barbituric acid and derivatives thereof, xanthine and derivatives thereof, phosphonic acids, and phosphates. Similarly, in certain embodiments, a chargeable monomeric unit that is chargeable to an anionic monomeric unit useful herein is a monomeric unit that comprises a carboxylic acid, anhydride, sulfonamide, sulfonic acid, sulfinic acid, sulfuric acid, phosphoric acid, phosphinic acid, boronic acid, phosphorous acid, or the like. In specific embodiments, an anionic chargeable monomeric unit useful herein is a (C₂-C₃)alkylacrylic acid-derived monomeric unit.

In some embodiments, the hydrophobic monomeric unit comprises a hydrophobic group such as but not limited to an alkyl group, a heteroalkyl group, an aryl group, a heteroaryl group. In some embodiments, the hydrophobic monomeric unit is, by way of non-limiting example, a butyl methacrylate, butyl acrylate, styrene, or the like. In specific embodiments, hydrophobic monomeric unit useful herein is a monomeric unit derived from (C₂-C₈)alkyl ester of (C₂-C₈)alkylacrylic acid.

### RNAi Agent Conjugation

In some embodiments, the polymer bioconjugates provided herein are useful for delivering RNAi agents (e.g., siRNA) to an individual in need thereof. In certain of such embodiments provided herein is a polymer bioconjugate comprising an RNAi agent conjugated to an end of a pH-dependent membrane-destabilizing polymer. In more specific embodiments, the RNAi agent is conjugated to the alpha end of the pH-dependent membrane-destabilizing polymer, and in other specific embodiments, the RNAi agent is conjugated to the omega end of the polymer.

In some embodiments, the RNAi molecule is a polynucleotide. In certain embodiments, the polynucleotideis an oligonucleotide gene expression modulator. In further embodiments, the polynucleotide is an oligonucleotide knockdown agent or the RNAi agent. In specific embodiments, the polynucleotide is a dicer substrate or siRNA.

In certain embodiments, the polynucleotide comprises 5′ and a 3′ end and is coupled to the membrane-destabilizing polymer at either the 5′ or 3′ end of the polynucleotide. In various embodiments, RNAi agent is covalently coupled to the membrane-destabilizing polymer through a linking moiety.

In some embodiments, the linking moiety comprises an affinity binder pair. In certain embodiments, a polynucleotide and/or one of the ends of the pH-dependent membrane destabilizing polymer is modified with chemical moieties that afford a polynucleotide and/or a polymer that have an affinity for one another, such as arylboronic acid-salicylhydroxamic acid, leucine zipper or other peptide motifs, or other types of chemical affinity linkages.

In some embodiments, an RNAi agent (e.g., oligonucleotide) is chemically coupled to the pH-dependent membrane destabilizing polymer of the polymer bioconjugate by any suitable chemical conjugation technique. In some embodiments, polymer bioconjugates comprising an RNAi agent are formed by conjugation of the RNAi agent with the conjugatable moiety at the alpha end of the pH-dependent membrane destabilizing polymer. In some embodiments, polymer bioconjugates comprising an RNAi agent are formed by conjugation of the RNAi agent with the conjugatable moiety at the omega end of the pH-dependent membrane destabilizing polymer. The linking moiety (e.g., a covalent bond) between a polymer and an RNAi agent of a polymer bioconjugate described herein is, optionally, non-cleavable, or cleavable. In certain embodiments, a precursor of an RNAi agent (e.g. a dicer substrate) is attached to the polymer (e.g., the alpha or omega end conjugatable group of the polymer) by a non-cleavable linking moiety. In some embodiments, an RNAi agent is attached through a cleavable linking moiety. In some instances, the linking moiety between the RNAi agent and the polymer of the polymer bioconjugate provided herein comprises a cleavable bond. In other instances, the linking moiety between the RNAi agent and the polymer of the polymer bioconjugate provided herein is non-cleavable. In certain embodiments, the cleavable bonds utilized in the polymer bioconjugates described herein include, by way of non-limiting example, disulfide bonds (e.g., disulfide bonds that dissociate in the reducing environment of the cytoplasm). In some embodiments, the linking moiety is cleavable and/or comprises a bond that is cleavable in endosomal conditions. In some embodiments, the linking moiety is cleavable and/or comprises a bond that is cleavable by a specific enzyme (e.g., a phosphatase, or a protease). In some embodiments, the linking moiety is cleavable and/or comprises a bond that is cleavable upon a change in an intracellular parameter (e.g., pH, redox potential). In some embodiments, covalent association between a polymer (e.g., the alpha or omega end conjugatable group of the polymer) and an RNAi agent (e.g., an oligonucleotide or siRNA) is achieved through any suitable chemical conjugation method, including but not limited to amine-carboxyl linkers, amine-aldehyde linkers, amine-ketone linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In some embodiments, a bifunctional cross-linking reagent is employed to achieve the covalent conjugation between suitable conjugatable groups of RNAi agent and pH-dependent membrane destabilizing polymer. In some embodiments, conjugation is also performed with pH-sensitive bonds and linkers, including, but not limited to, hydrazone and acetal linkages. In certain embodiments, an RNAi (e.g., a ribooligonucleotide) molecule is covalently linked to a boronic acid functionality (e.g., a phenylboronic acid residue) incorporated into the alpha or the omega end of the polymer through the formation of an ester of the boronic acid with the 2′ and 3′-hydroxyl of the terminal ribose residue of the RNAi agent. Any other suitable conjugation method is optionally utilized as well, for example a large variety of conjugation chemistries are available (see, for example, Bioconjugation, Aslam and Dent, Eds, Macmillan, 1998 and chapters therein).

In some embodiments, an RNAi agent (e.g., oligonucleotide) is chemically coupled to a pendant group of a monomeric unit of the pH-dependent membrane destabilizing polymer of the polymer bioconjugate by any suitable chemical conjugation technique. In some embodiments, polymer bioconjugates comprising an RNAi agent are formed by conjugation of the RNAi agent with the conjugatable moiety of the pendant group of the pH-dependent membrane destabilizing polymer. The linking moiety (e.g., a covalent bond) between a polymer and an RNAi agent of a polymer bioconjugate described herein is, optionally, non-cleavable, or cleavable. In some embodiments, an RNAi agent is attached through a cleavable linking moiety. In some instances, the linking moiety between the RNAi agent and the polymer of the polymer bioconjugate provided herein comprises a cleavable bond. In other instances, the linking moiety between the RNAi agent and the polymer of the polymer bioconjugate provided herein is non-cleavable. In some embodiments, the linking moiety is cleavable and/or comprises a bond that is cleavable in endosomal conditions. In some embodiments, the linking moiety is cleavable and/or comprises a bond that is cleavable by a specific enzyme (e.g., a phosphatase, or a protease. In some embodiments, covalent association between a polymer and an RNAi agent (e.g., an oligonucleotide or siRNA) is achieved through any suitable chemical conjugation method. In some embodiments, an RNAi agent (e.g., oligonucleotide) is chemically coupled to a monomeric unit of the pH-dependent membrane destabilizing polymer comprising a conjugatable group.

In certain embodiments, a conjugate of an RNAi agent (e.g., oligonucleotide) with a pH-dependent membrane destabilizing polymer (e.g., the alpha or omega end conjugatable group of the polymer) of the polymer bioconjugate provided herein is prepared according to a process comprising the following two steps: (1) activating a modifiable end group (for example, 5′- or 3′-hydroxyl or amino group) of an oligonucleotide using any suitable activation reagents, such as but not limited tol-ethyl-3,3-dimethylaminopropyl carbodiimide (EDAC), imidazole, N-hydrosuccinimide (NHS) and dicyclohexylcarbodiimide (DCC), HOBt (1-hydroxybenzotriazole), p-nitrophenylchloroformate, carbonyldiimidazole (CDI), and N,N′-disuccinimidyl carbonate (DSC); and (2) covalently linking the polymer (e.g., the alpha or omega end of the polymer) to the end of the oligonucleotide. In some embodiments, the 5′- or 3′-end modifiable group of an oligonucleotide is substituted by other functional groups prior to conjugation with the polymer. For example, hydroxyl group (—OH) is optionally substituted with a linker carrying sulfhydryl group (—SH), carboxyl group (—COOH), or amine group (—NH₂).

In yet another embodiment, an oligonucleotide comprising a functional group introduced into one or more of the bases (for example, a 5-aminoalkylpyrimidine), is conjugated to a polymer of the polymer bioconjugate provided herein using a an activating agent or a reactive bifunctional linker according to any suitable procedure. A variety of such activating agents and bifunctional linkers is available commercially from such suppliers as Sigma, Pierce, Invitrogen and others.

In some specific embodiments, the pH-dependent membrane destabilizing polymer is prepared by RAFT polymerization employing a chain-transfer agent comprising a masked conjugatable group. In a specific instance, pyridyl-disulfide comprising CTA is used to synthesize such polymer. The covalent end-conjugation of an RNAi agent is achieved by treating a thiol-comprising RNAi agent with the polymer. In some instances, an excess of a thiol-comprising RNAi agent compared to polymer concentration is used to achieve the conjugation.

In certain embodiments, copolymer bioconjugates described herein facilitate intracellular delivery of a biomolecular agent (e.g., an antibody, siRNA or the like). In certain embodiments, copolymer bioconjugates described herein facilitate intracellular delivery of siRNA that is connected by direct polymer-RNA conjugation. In certain embodiments, a copolymer bioconjugate that enhances intracellular delivery of siRNA comprises a first block that enhances water solubility (e.g., a first block that comprises hydrophilic monomers) and/or pharmacokinetic properties, and a block that is pH-responsive. In some of such embodiments, a copolymer bioconjugate that enhances intracellular delivery of siRNA comprises an end-functionalized diblock copolymer that incorporates an N-(2-hydroxypropyl)methacrylamide (HPMA) first block (e.g., to enhance water solubility and/or pharmacokinetic properties), and a pH-responsive polymer block comprising dimethylaminoethyl methacrylate (DMAEMA), propylacrylic acid (PAA), and butyl methacrylate (BMA) residues (e.g., to provide a mechanism for siRNA endosomal escape). A diblock copolymer that enhances intracellular delivery of siRNA polymer is prepared using any suitable technique, such as one described herein (e.g., reversible addition fragmentation chain transfer (RAFT)). In some embodiments, an end-functionalized diblock copolymer carrier is conjugated to a thiolated siRNA via any technique, such as one described herein (e.g., a reducible disulfide bond that ensures polymer release of the RNA upon delivery to the cytosol).

### Targeting Ligands

In certain embodiments, polymer bioconjugates described herein comprise at least one targeting ligand (e.g., a moiety that targets a specific cell or type of cell). In certain embodiments, polymer bioconjugates described herein comprise a plurality of small molecule targeting ligands (e.g., carbohydrate moieties). In some embodiments, one or more targeting ligands are coupled to the membrane-destabilizing polymer at the alpha end or at the omega end opposing the RNAi agent, or to a pendant group of one or more monomeric units of the membrane-destabilizing polymer. In specific instances, the polymer bioconjugates provided herein are useful for delivery of therapeutic RNAi agents to specifically targeted cells of an individual. In certain instances, the efficiency of the cell uptake of the polymer bioconjugates is enhanced by incorporation of targeting moieties into the polymer bioconjugate. A “targeting ligand” (used interchangeably with “targeting moiety”) is any affinity reagent which recognizes the surface of a cell (e.g., a select cell). In some embodiments, targeting moieties recognize a cell surface antigen or bind to a receptor on the surface of the target cell. Suitable targeting ligands include, by way of non-limiting example, antibodies, antibody-like molecules, or peptides, such as an integrin-binding peptides such as RGD-containing peptides, or small molecules, such as vitamins, e.g., folate, sugars such as lactose and galactose, or other small molecules. Cell surface antigens include a cell surface molecule such as a protein, sugar, lipid or other antigen on the cell surface. In specific embodiments, the cell surface antigen undergoes internalization. Examples of cell surface antigens targeted by the targeting moieties of the polymer bioconjugates provided herein include, but are not limited, to the transferrin receptor type 1 and 2, the EGF receptor, HER2/Neu, VEGF receptors, integrins, NGF, CD2, CD3, CD4, CD8, CD19, CD20, CD22, CD33, CD43, CD38, CD56, CD69, and the asialoglycoprotein receptor. A targeting ligand can also comprise an artificial affinity molecule, e.g., a peptidomimetic or an aptamer.

Targeting ligands are attached, in various embodiments, to either end of a polymer (e.g., block copolymer) of the polymer bioconjugate, or to a side chain or a pendant group of a monomeric unit, or incorporated into a polymer. In some instances, the targeting ligand is covalently coupled to the membrane-destabilizing chargeable polymer at the opposite end from the RNAi agent. In certain embodiments, a monomer comprising a targeting agent residue (e.g, a polymerizable derivative of a targeting agent such as an (alkyl)acrylic acid derivative of a peptide) is co-polymerized to form the copolymer forming the polymer bioconjugate provided herein. In certain embodiments, one or more targeting ligand is coupled to the membrane-destabilizing polymer of the polymer bioconjugate provided herein through a linking moiety. In some embodiments, the linking moiety coupling the targeting ligand to the membrane-destabilizing polymer is a cleavable linking moiety (e.g., comprises a cleavable bond). In some embodiments, the linking moiety is cleavable and/or comprises a bond that is cleavable in endosomal conditions. In some embodiments, the linking moiety is cleavable and/or comprises a bond that is cleavable by a specific enzyme (e.g., a phosphatase, or a protease). In some embodiments, the linking moiety is cleavable and/or comprises a bond that is cleavable upon a change in an intracellular parameter (e.g., pH, redox potential).

In some embodiments, the targeting agent is a proteinaceous targeting agent (e.g., a peptide, and antibody, an antibody fragment). In some specific embodiments, a plurality of polymer bioconjugates comprising an RNAi agent coupled to an end of a pH-dependent membrane destabilizing polymer is covalently coupled to the proteinaceous targeting ligand. In some instances, a single proteinaceous targeting ligand conjugate is used to deliver one or more RNAi agents coupled to an end of a pH-dependent membrane destabilizing polymer bioconjugates to a cell. In some instances, at least 2, at least 5, or at least 10 polymer bioconjugates are covalently coupled to the proteinaceous targeting ligand.

Attachment of the targeting moiety to the polymer is achieved in any suitable manner, e.g., by any one of a number of conjugation chemistry approaches including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In specific embodiments, “click” chemistry is used to attach the targeting ligand to the polymers of the polymer bioconjugates provided herein (for example of “click” reactions, see Wu, P.; Fokin, V. V. Catalytic Azide-Alkyne Cycloaddition: Reactivity and Applications. Aldrichim. Acta 2007, 40, 7-17). A large variety of conjugation chemistries are optionally utilized (see, for example, Bioconjugation, Aslam and Dent, Eds, Macmillan, 1998 and chapters therein). In some embodiments, targeting ligands are attached to a monomer and the resulting compound is then used in the polymerization synthesis of a polymer (e.g., copolymer) utilized in a polymer bioconjugate described herein. In some embodiments, the targeting ligand is attached to the sense or antisense strand of siRNA bound to a polymer of the polymer bioconjugate. In certain embodiments, the targeting agent is attached to a 5′ or a 3′ end of the sense or the antisense strand.

In specific embodiments, the polymer bioconjugates provided herein are biocompatible. As used herein, “biocompatible” refers to a property of a compound (e.g., polymer bioconjugate) characterized by it, or its in vivo degradation products, being not, or at least minimally and/or reparably, injurious to living tissue; and/or not, or at least minimally and controllably, causing an immunological reaction in living tissue. With regard to salts, it is presently preferred that both the cationic and the anionic species be biocompatible. As used herein, “physiologically acceptable” is interchangeable with biocompatible. In some instances, the polymer bioconjugates and polymers used therein (e.g., copolymers) exhibit low toxicity compared to cationic lipids.

In some instances, pH-dependent membrane destabilizing polymers utilized in the polymer bioconjugates described herein comprise polyethyleneglycol (PEG) chains or blocks with molecular weights of approximately from 1,000 to approximately 30,000. In some embodiments, PEG is conjugated to polymer ends groups, or to one or more pendant conjugatable group present in a polymer of a polymer bioconjugate provided herein. In certain embodiments, a monomer comprising a PEG residue is co-polymerized to form the copolymer forming the polymer bioconjugate provided herein.

### Cell Uptake

In some embodiments, the polymer bioconjugates comprising RNAi agents (e.g., oligonucleotides or siRNA) are delivered to cells by endocytosis. Intracellular vesicles and endosomes are used interchangeably throughout this specification. Successful delivery of RNAi agents (e.g., oligonucleotide or siRNA) into the cytoplasm generally has a mechanism for endosomal escape. In certain instances, the polymer bioconjugates comprising RNAi agents (e.g., oligonucleotide or siRNA) provided herein are sensitive to the lower pH in the endosomal compartment upon endocytosis. In certain instances, endocytosis triggers protonation or charge neutralization of chargeable monomeric units or species chargeable to anionic units (e.g., propyl acrylic acid units) or species of the polymers and/or polymer bioconjugates provided herein, resulting in a conformational transition in the polymer. In certain instances, this conformational transition results in a more hydrophobic membrane destabilizing form which mediates release of the therapeutic agent (e.g., oligonucleotide or siRNA) from the endosomes to the cytoplasm. In those polymer conjugates comprising siRNA, delivery of siRNA into the cytoplasm allows its mRNA knockdown effect to occur. In those polymer conjugates comprising other types of RNAi agents, delivery into the cytoplasm allows their desired action to occur.

### Therapy

In some embodiments, the polymer bioconjugates provided herein are useful in treating a subject at risk for or afflicted with disorders associated with and/or caused by high plasma levels or cholesterol, apolipoprotein b, and/or LDL cholesterol, e.g. hypercholesterolemia. The treatment comprises providing a polymer bioconjugate comprising an RNAi agent (e.g., an oligonucleotide agent), wherein the RNAi agent silences (e.g., by cleavage) a gene or a gene product which promotes such condition. In some embodiments the RNAi agent silences proprotein convertase subtilisin/kexin type 9 (PCSK9) gene responsible for regulation of low density lipoprotein (LDLR) levels and function, and thus polymer bioconjugates comprising such RNAi agent is used to treat a subject having or at risk for a disorder characterized by unwanted PCSK9 expression, e.g., disorders associated with and/or caused by high plasma levels or cholesterol, apolipoprotein b, and/or LDL cholesterol, e.g. hypercholesterolemia. In some embodiments, the polymer bioconjugates deliver PCSK9-silencing RNAi agent (e.g, siRNA) to a cell expressing PCSK9. In some embodiments, the cell is a liver cell.

In some embodiments, the polymer bioconjugates provided herein are useful in treating a subject at risk for or afflicted with unwanted cell proliferation (e.g., malignant or nonmalignant cell proliferation). The treatment comprises providing a polymer bioconjugate comprising an RNAi agent (e.g., an oligonucleotide agent), wherein the RNAi agent silences (e.g., by cleavage) a gene or a gene product which promotes unwanted cell proliferation; and administering a therapeutically effective dose of the polymer bioconjugates to a subject (e.g., a human subject.) In some embodiments, the RNAi agent is a polynucleotide (e.g., an oligonucleotide) which is homologous to and silences (e.g., by cleavage) a gene.

In certain embodiments, the gene is but is not limited to a growth factor or growth factor receptor gene, a phosphatase, a kinase, e.g., a protein tyrosine, serine or threonine kinase gene, an adaptor protein gene, a gene encoding a G protein superfamily molecule, or a gene encoding a transcription factor. In some instances, the polymer bioconjugate comprises an RNAi agent which silences a gene which is expressed in a specific tissue or organ, including, but not limited to lung, pancreas, liver, kidney, ovary, muscle, skin, breast, colon, stomach, and the like.

In some embodiments, the RNAi agent silences one or more of the following genes: the PDGF beta gene, and thus is used to treat a subject having or at risk for a disorder characterized by unwanted PDGF beta expression, e.g., testicular and lung cancers; an Erb-B gene (e.g., Erb-B-2 or Erb-B-3), and thus is used to treat a subject having or at risk for a disorder characterized by unwanted Erb-B expression, e.g., breast or lung cancer; the Src gene, and thus is used to treat a subject having or at risk for a disorder characterized by unwanted Src expression, e.g., colon cancers; the CRK gene, and thus is used to treat a subject having or at risk for a disorder characterized by unwanted CRK expression, e.g., colon and lung cancers; the GRB2 gene, and thus is used to treat a subject having or at risk for a disorder characterized by unwanted GRB2 expression, e.g., squamous cell carcinoma; the RAS gene, and thus is used to treat a subject having or at risk for a disorder characterized by unwanted RAS expression, e.g., pancreatic, colon and lung cancers, and chronic leukemia; the MEKK gene, and thus is used to treat a subject having or at risk for a disorder characterized by unwanted MEKK expression, e.g., squamous cell carcinoma, melanoma or leukemia; the JNK gene, and thus is used to treat a subject having or at risk for a disorder characterized by unwanted JNK expression, e.g., pancreatic or breast cancers; the RAF gene, and thus is used to treat a subject having or at risk for a disorder characterized by unwanted RAF expression, e.g., lung cancer or leukemia; the Erk1/2 gene, and thus is used to treat a subject having or at risk for a disorder characterized by unwanted Erk1/2 expression, e.g., lung cancer; the PCNA(p21) gene, and thus is used to treat a subject having or at risk for a disorder characterized by unwanted PCNA expression, e.g., lung cancer; the MYB gene, and thus is used to treat a subject having or at risk for a disorder characterized by unwanted MYB expression, e.g., colon cancer or chronic myelogenous leukemia; the c-MYC gene, and thus is used to treat a subject having or at risk for a disorder characterized by unwanted c-MYC expression, e.g., Burkitt's lymphoma or neuroblastoma; the JUN gene, and thus is used to treat a subject having or at risk for a disorder characterized by unwanted JUN expression, e.g., ovarian, prostate or breast cancers; the FOS gene, and thus is used to treat a subject having or at risk for a disorder characterized by unwanted FOS expression, e.g., skin or prostate cancers; the BCL-2 gene, and thus is used to treat a subject having or at risk for a disorder characterized by unwanted BCL-2 expression, e.g., lung or prostate cancers or Non-Hodgkin lymphoma; the Cyclin D gene, and thus is used to treat a subject having or at risk for a disorder characterized by unwanted Cyclin D expression, e.g., esophageal and colon cancers; the VEGF gene, and thus is used to treat a subject having or at risk for a disorder characterized by unwanted VEGF expression, e.g., esophageal and colon cancers; the EGFR gene, and thus is used to treat a subject having or at risk for a disorder characterized by unwanted EGFR expression, e.g., breast cancer; the Cyclin A gene, and thus is used to treat a subject having or at risk for a disorder characterized by unwanted Cyclin A expression, e.g., lung and cervical cancers; the Cyclin E gene, and thus is used to treat a subject having or at risk for a disorder characterized by unwanted Cyclin E expression, e.g., lung and breast cancers; the WNT-1 gene, and thus is used to treat a subject having or at risk for a disorder characterized by unwanted WNT-1 expression, e.g., basal cell carcinoma; the beta-catenin gene, and thus is used to treat a subject having or at risk for a disorder characterized by unwanted beta-catenin expression, e.g., adenocarcinoma or hepatocellular carcinoma; the c-MET gene, and thus is used to treat a subject having or at risk for a disorder characterized by unwanted c-MET expression, e.g., hepatocellular carcinoma; the PKC gene, and thus is used to treat a subject having or at risk for a disorder characterized by unwanted PKC expression, e.g., breast cancer; the NFKB gene, and thus is used to treat a subject having or at risk for a disorder characterized by unwanted NFKB expression, e.g., breast cancer; the STAT3 gene, and thus is used to treat a subject having or at risk for a disorder characterized by unwanted STAT3 expression, e.g., prostate cancer; the survivin gene, and thus is used to treat a subject having or at risk for a disorder characterized by unwanted survivin expression, e.g., cervical or pancreatic cancers; the Her2/Neu gene, and thus is used to treat a subject having or at risk for a disorder characterized by unwanted Her2/Neu expression, e.g., breast cancer; the topoisomerase I gene, and thus is used to treat a subject having or at risk for a disorder characterized by unwanted topoisomerase I expression, e.g., ovarian and colon cancers; the topoisomerase II alpha gene, and thus is used to treat a subject having or at risk for a disorder characterized by unwanted topoisomerase II expression, e.g., breast and colon cancers.

In other embodiments the RNAi agent silences mutations in one of the following genes: the p73 gene, and thus is used to treat a subject having or at risk for a disorder characterized by unwanted p73 expression, e.g., colorectal adenocarcinoma; the p21(WAF1/CIP1) gene, and thus is used to treat a subject having or at risk for a disorder characterized by unwanted p21(WAF1/CIP1) expression, e.g., liver cancer; the p27(KIP1) gene, and thus is used to treat a subject having or at risk for a disorder characterized by unwanted p27(KIP1) expression, e.g., liver cancer; the PPM1D gene, and thus is used to treat a subject having or at risk for a disorder characterized by unwanted PPM1D expression, e.g., breast cancer; the RAS gene, and thus is used to treat a subject having or at risk for a disorder characterized by unwanted RAS expression, e.g., breast cancer; the caveolin I gene, and thus is used to treat a subject having or at risk for a disorder characterized by unwanted caveolin I expression, e.g., esophageal squamous cell carcinoma; the MIB I gene, and thus is used to treat a subject having or at risk for a disorder characterized by unwanted MIB I expression, e.g., male breast carcinoma (MBC); MTAI gene, and thus is used to treat a subject having or at risk for a disorder characterized by unwanted MTAI expression, e.g., ovarian carcinoma; the M68 gene, and thus is used to treat a subject having or at risk for a disorder characterized by unwanted M68 expression, e.g., human adenocarcinomas of the esophagus, stomach, colon, and rectum.

In some embodiments the RNAi agent silences mutations in tumor suppressor genes, and thus is used as a method to promote apoptotic activity in combination with chemotherapeutics. In some embodiments the in the tumor suppressor gene is selected from one or more of the following tumor suppressor genes: the p53 tumor suppressor gene, the p53 family member DN-p63, the pRb tumor suppressor gene, the APC1 tumor suppressor gene, the BRCA1 tumor suppressor gene, the PTEN tumor suppressor gene.

In some embodiments the RNAi agent silences one of the following fusion genes: mLL fusion genes, e.g., mLL-AF9, and thus is used to treat a subject having or at risk for a disorder characterized by unwanted mLL fusion gene expression, e.g., acute leukemias; the BCR/ABL fusion gene, and thus is used to treat a subject having or at risk for a disorder characterized by unwanted BCR/ABL fusion gene expression, e.g., acute and chronic leukemias; the TEL/AML1 fusion gene, and thus is used to treat a subject having or at risk for a disorder characterized by unwanted TEL/AML1 fusion gene expression, e.g., childhood acute leukemia; the EWS/FLI1 fusion gene, and thus is used to treat a subject having or at risk for a disorder characterized by unwanted EWS/FLI1 fusion gene expression, e.g., Ewing Sarcoma; the TLS/FUS1 fusion gene, and thus is used to treat a subject having or at risk for a disorder characterized by unwanted TLS/FUS1 fusion gene expression, e.g., Myxoid liposarcoma; the PAX3/FKHR fusion gene, and thus is used to treat a subject having or at risk for a disorder characterized by unwanted PAX3/FKHR fusion gene expression, e.g., Myxoid liposarcoma; the AML1/ETO fusion gene, and thus is used to treat a subject having or at risk for a disorder characterized by unwanted AML1/ETO fusion gene expression, e.g., acute leukemia.

In some aspects herein the polymer bioconjugates provide therapeutic agents for treating a subject, e.g., a human, at risk for or afflicted with a disease or disorder that may benefit by angiogenesis inhibition e.g., cancer or retinal degeneration. The treatment comprises providing a polymer bioconjugate comprising an RNAi agent (e.g., an oligonucleotide), wherein said oligonucleotide agent is homologous to and/or can silence, e.g., by cleavage, a gene which mediates angiogenesis (e.g., VEGF-R1, VEGF-R2 or a gene encoding signaling proteins for these receptors' pathways); and administering a therapeutically effective dosage of said polymer bioconjugate comprising the oligonucleotide agent to a subject, e.g., a human subject.

In some embodiments the RNAi agent silences one of the following genes: the alpha v-integrin gene, and thus is used to treat a subject having or at risk for a disorder characterized by unwanted alpha V integrin, e.g., brain tumors or tumors of epithelial origin; the Flt-1 receptor gene, and thus is used to treat a subject having or at risk for a disorder characterized by unwanted Flt-1 receptors, e.g., cancer and rheumatoid arthritis; the tubulin gene, and thus is used to treat a subject having or at risk for a disorder characterized by unwanted tubulin, e.g., cancer and retinal neovascularization.

In some aspects the polymer bioconjugates comprising RNAi agents provided herein relate to a method of treating a subject infected with a virus or at risk for or afflicted with a disorder or disease associated with a viral infection. The method comprises providing a polymer bioconjugate comprising an RNAi agent (e.g., an oligonucleotide agent), wherein said oligonucleotide agent is homologous to and/or can silence, e.g., by cleavage, a viral gene or a cellular gene which mediates viral function, e.g., entry or growth; and administering a therapeutically effective dose of said oligonucleotide agent to a subject, e.g., a human subject.

In some embodiments, the polymer bioconjugates comprising an RNAi agent are useful in treatment of subjects infected with the Human Papilloma Virus (HPV) or at risk for or afflicted with a disorder mediated by HPV, e.g, cervical cancer.

In some embodiments, the polymer bioconjugate comprises an RNAi agent silencing expression of a HPV gene. In some embodiments, the HPV gene is selected from the group of E2, E6, or E7.

In another embodiment the expression of a human gene that is required for HPV replication is reduced.

In some embodiments, the polymer bioconjugate comprises an RNAi agent useful in treating patients infected by the Human Immunodeficiency Virus (HIV) or at risk for or afflicted with a disorder mediated by HIV, e.g., Acquired Immune Deficiency Syndrome (AIDS). In some embodiments, the expression of an HIV gene is reduced. In other embodiments, the HIV gene is CCR5, Gag, or Rev. In some embodiments the expression of a human gene that is required for HIV replication is reduced. In some embodiments, the gene is CD4 or Tsg101.

In some embodiments, the polymer bioconjugate comprises an RNAi agent useful for treating patients infected by the Hepatitis B Virus (HBV) or at risk for or afflicted with a disorder mediated by HBV, e.g., cirrhosis and heptocellular carcinoma. In one embodiment, the expression of a HBV gene is reduced. In other embodiment, the targeted HBV gene encodes one of the groups of the tail region of the HBV core protein, the pre-cregious (pre-c) region, or the cregious (c) region. In other embodiments a targeted HBV-RNA sequence is comprised of the poly(A) tail. In some embodiments the expression of a human gene that is required for HBV replication is reduced.

In some embodiments, the polymer bioconjugate comprises an RNAi agent useful for treating patients infected with, or at risk for or afflicted with a disorder mediated by a virus selected from the following viruses: the Hepatitis A Virus (HAV); Hepatitis C Virus (HCV); any of the group of Hepatitis Viral strains comprising hepatitis D, E, F, G, or H; the Respiratory Syncytial Virus (RSV); the herpes Cytomegalovirus (CMV); the herpes Epstein Barr Virus (EBV); Kaposi's Sarcoma-associated Herpes Virus (KSHV); the JC Virus (JCV); myxovirus (e.g., virus causing influenza), rhinovirus (e.g., virus causing the common cold), or coronavirus (e.g., virus causing the common cold); the St. Louis Encephalitis flavivirus; the Tick-borne encephalitis flavivirus; the Murray Valley encephalitis flavivirus; the dengue flavivirus; the Simian Virus 40 (SV40); the encephalomyocarditis virus (EMCV); the measles virus (MV); the Varicella zoster virus (VZV); an adenovirus (e.g. virus causing a respiratory tract infection); the poliovirus; or a poxvirus (a poxvirus causing smallpox). In some embodiments the expression of a human gene that is required for the replication of these viruses is reduced.

In some embodiments, the polymer bioconjugate comprises an RNAi agent useful for treating patients infected by the Herpes Simplex Virus (HSV) or at risk for or afflicted with a disorder mediated by HSV, e.g, genital herpes and cold sores as well as life-threatening or sight-impairing disease, e.g., mainly in immunocompromised patients. In some embodiments, the expression of a HSV gene is reduced. In other embodiment, the targeted HSV gene encodes DNA polymerase or the helicase-primase. In some embodiments the expression of a human gene that is required for HSV replication is reduced.

In some embodiments, the polymer bioconjugate comprises an RNAi agent useful for treating patients infected by the West Nile Virus or at risk for or afflicted with a disorder mediated by West Nile Virus. In some embodiments, the expression of a West Nile Virus gene is reduced. In other preferred embodiments, the West Nile Virus gene is selected from the group comprising E, NS3, or NS5. In some embodiments the expression of a human gene that is required for West Nile Virus replication is reduced.

In some embodiments, the polymer bioconjugate comprises an RNAi agent useful for treating patients infected by the Human T Cell Lymphotropic Virus (HTLV), or a disease or disorder associated with this virus, e.g., leukemia or myelopathy. In some embodiments, the expression of a HTLV gene is reduced. In some embodiments, the HTLV1 gene is the Tax transcriptional activator. In some embodiments, the expression of a human gene that is required for HTLV replication is reduced.

In some aspects, the polymer bioconjugate comprises an RNAi agent useful for treating a subject infected with a pathogen, e.g., a bacterial, amoebic, parasitic, or fungal pathogen. The method of treatment comprises providing a polymer bioconjugate comprising an RNAi agent, wherein said RNAi agent is homologous to and/or can silence, e.g., by cleavage of a pathogen gene or a gene involved in the pathogen's growth; and administering a therapeutically effective dose of said RNAi agent to a subject, e.g., a human subject. The target gene is selected from a gene involved in the pathogen's growth, cell wall synthesis, protein synthesis, transcription, energy metabolism, e.g., the Krebs cycle, or toxin production.

Thus, in some embodiments, the polymer bioconjugate comprises an RNAi agent useful for of treating patients infected by a plasmodium that causes malaria. In some embodiments, the expression of a plasmodium gene is reduced. In other embodiments, the gene is apical membrane antigen 1 (AMA1). In some embodiments, the expression of a human gene that is required for plasmodium replication is reduced.

In some embodiments, the polymer bioconjugate comprises an RNAi agent useful for treating patients infected by Mycobacterium ulcerans, Mycobacterium tuberculosis, Mycobacterium leprae, Staphylococcus aureus, Streptococcus pneumoniae, Streptococcus pyogenes, Chlamydia pneumoniae, Mycoplasma pneumoniae, or a disease or disorder associated with any of these pathogens. In some embodiments, the expression of a bacterial gene and/or a human gene that is required for the replication of these bacteria is reduced.

In some embodiments, the diseases treated by the polymer bioconjugates provided herein may be systemic or present in a specific tissue, e.g., the lung, skin, liver, breast, kidney, pancreas, CNS, or the like. In certain aspects, the RNAi agent silences a gene which mediates or is involved in a metabolic disease or disorder, e.g., diabetes, obesity, and the like. In certain embodiments, the RNAi agent silences a gene which mediates or is involved in a pulmonary disease or disorder, e.g., chronic obstructive pulmonary disease (COPD), cystic fibrosis, or lung cancer. In some aspects herein, the polymer bioconjugates comprise an RNAi agent useful for and/or related to a method of treating a subject, e.g., a human, at risk for or afflicted with a disease or disorder characterized by an unwanted immune response, e.g., an inflammatory disease or disorder or an autoimmune disease or disorder. The method comprises providing a polymer bioconjugate comprising an RNAi agent, wherein said RNAi agent is homologous to and/or can silence, e.g., by cleavage, a gene which mediates an unwanted immune response; and administering said oligonucleotide agent to a subject, e.g., a human subject. In some embodiments, the disease or disorder is an ischemia or reperfusion injury, e.g., ischemia or reperfusion injury associated with acute myocardial infarction, unstable angina, cardiopulmonary bypass, surgical intervention e.g., angioplasty, e.g., percutaneous transluminal coronary angioplasty, the response to a transplantated organ or tissue, e.g., transplanted cardiac or vascular tissue; or thrombolysis. In other embodiments, the disease or disorder is restenosis, e.g., restenosis associated with surgical intervention e.g., angioplasty, e.g., percutaneous transluminal coronary angioplasty. In other embodiments, the disease or disorder is Inflammatory Bowel Disease, e.g., Crohn Disease or Ulcerative Colitis. In some embodiments, the disease or disorder is inflammation associated with an infection or injury. In other embodiments, the disease or disorder is asthma, allergy, lupus, multiple sclerosis, diabetes, e.g., type II diabetes, arthritis, e.g., rheumatoid or psoriatic. In certain embodiments the RNAi agent silences an integrin or co-ligand thereof, e.g., VLA4, VCAM, ICAM. In other embodiments the oligonucleotide agent silences a selectin or co-ligand thereof, e.g., P-selectin, E-selectin (ELAM), 1-selectin, P-selectin glycoprotein-1 (PSGL-1). In certain embodiments the RNAi agent silences a component of the complement system, e.g., C3, C5, C3aR, C5aR, C3 convertase, and C5 convertase. In some embodiments the RNAi agent silences a chemokine or receptor thereof, e.g., TNFI, TNFJ, IL-1J, 1L-2, IL-2RI, IL-4, IL-4R, IL-5, IL-6, IL-8, TNFRI, TNFRII, IgE, SCYA11, and CCR3. In other embodiments the RNAi agent silences GCSF, Gro1, Gro2, Gro3, PF4, MIG, Pro-Platelet Basic Protein (PPBP), MIP-1I, MIP-1J, RANTES, MCP-1, MCP-2, MCP-3, CMBKR1, CMBKR2, CMBKR3, CMBKR5, AIF-1, or I-309.

In some aspects, the polymer bioconjugates comprise an RNAi agent useful for treating a subject, e.g., a human, at risk for or afflicted with a neurological disease or disorder. The method comprises providing a polymer bioconjugate comprising an RNAi agent, wherein said RNAi agent is homologous to and/or can silence, e.g., by cleavage, a gene which mediates a neurological disease or disorder; and administering a therapeutically effective dose of said RNAi agent to a subject, e.g., a human. In some embodiments the disease or disorder is Alzheimer Disease or Parkinson Disease. In certain embodiments the oligonucleotide agent silences an amyloid-family gene, e.g., APP; a presenilin gene, e.g., PSEN1 and PSEN2, or I-synuclein. In other embodiments the disease or disorder is a neurodegenerative trinucleotide repeat disorder, e.g., Huntington disease, dentatorubral pallidoluysian atrophy or a spinocerebellar ataxia, e.g., SCA1, SCA2, SCA3 (Machado-Joseph disease), SCA7 or SCA8. In some embodiments the RNAi agent silences HD, DRPLA, SCA1, SCA2, MJDI, CACNL1A4, SCA7, or SCA8.

In certain aspects, the polymer bioconjugates provided herein comprise an RNAi agent capable of cleaving or silencing more than one gene. In these embodiments the RNAi agent is selected so that it has sufficient homology to a sequence found in more than one gene, e.g. a sequence conserved between these genes. Thus in some embodiments an RNAi agent targeted to such sequences effectively silences the entire collection of genes. In some aspects, the polymer bioconjugates provided herein comprise two or more types of RNAi agent wherein the RNAi agent silence different genes of the same disease or different diseases.

Any RNAi agent described herein is attached to the polymer of the polymer bioconjugate (e.g., pH dependent membrane destabilizing polymers) in any suitable manner, e.g., any manner described herein.

### Pharmaceutical Compositions

Polymer bioconjugates provided herein (e.g., those attached to one or more RNAi agent therapeutic agent, such as one or more oligonucleotide) are optionally provided in a composition (e.g., pharmaceutically acceptable composition). In some embodiments, the polymer bioconjugates provided herein is administered to a patient in any suitable manner, e.g., with or without stabilizers, buffers, and the like, to form a pharmaceutical composition. In some embodiments, the polymer bioconjugates provided herein are formulated and used as tablets, capsules or elixirs for oral administration, suppositories for rectal administration, sterile solutions, suspensions or solutions for injectable administration, and any other suitable compositions.

Provided are pharmaceutically acceptable formulations of the polymer bioconjugates comprising at least one RNAi therapeutic agent described herein. These formulations include salts of the above compounds, e.g., acid addition salts, e.g., salts of hydrochloric, hydrobromic, acetic acid, and benzene sulfonic acid. A pharmacological composition or formulation refers to a composition or formulation in a form suitable for administration, e.g., systemic administration, into a cell or patient, including for example a human. Suitable forms, in part, depend upon the use or the route of entry, e.g., oral, transdermal, or by injection. Thus, in specific embodiments wherein the polymer bioconjugate comprises and is delivering a polynucleotide, the formulation is in a form that does not prevent the polymer bioconjugate and, more specifically, the polynucleotide (e.g., oligonucleotide or siRNA) from reaching a target cell with the polynucleotide intact and/or functional. For example, in certain embodiments, pharmacological compositions injected into the blood stream are soluble and/or dispersible. Moreover, pharmaceutical compositions described herein are, preferably, non-toxic. In some embodiments, wherein a polymer bioconjugate described herein is administered for therapeutic benefit, a therapeutic effective amount of the polymer bioconjugate comprising an RNAi therapeutic agent (e.g., a polynucleotide, such as an siRNA) is administered. In an exemplary embodiment, a therapeutically effective amount includes a sufficient amount of polymer bioconjugate to provide about 10 mg or less of siRNA per kg of individual.

In some embodiments, pharmaceutical compositions comprising a polymer bioconjugate, which comprise an RNAi therapeutic agent (e.g., a polynucleotide, such as an siRNA), are administered systemically. As used herein, “systemic administration” means in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. In some embodiments, the polymer bioconjugate compositions are administered topically.

In some embodiments, the compositions are prepared for storage or administration and include a pharmaceutically effective amount of the therapeutic agent comprising polymer bioconjugate in a pharmaceutically acceptable carrier or diluent. Any acceptable carriers or diluents are optionally utilized herein. Specific carriers and diluents and are described, e.g., in Remington's. Pharmaceutical Sciences, Mack Publishing Co., A. R. Gennaro Ed., 1985. For example, preservatives, stabilizers, dyes and flavoring agents are optionally added. These include sodium benzoate, sorbic acid and esters of p-hydroxybenzoic acid. In addition, antioxidants and suspending agents are optionally used. As used herein, the term “pharmaceutically acceptable carrier” means a non-toxic, inert solid, semi-solid or liquid filler, diluent, encapsulating material or formulation auxiliary of any type. Some examples of materials optionally used as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. In some embodiments, the pharmaceutical compositions provided herein are administered to humans and/or to animals, orally, rectally, parenterally, intracistemally, intravaginally, intranasally, intraperitoneally, topically (as by powders, creams, ointments, or drops), bucally, or as an oral or nasal spray.

In various embodiments, liquid dosage forms for oral administration include pharmaceutically acceptable emulsions, microemulsions, solutions, suspensions, syrups, and elixirs. In addition to the active ingredients (i.e., micelle-oligonucleotide complexes provided herein), the liquid dosage forms optionally further contain inert diluents or excipients, such as by way of non-limiting example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions optionally also include adjuvants such as wetting agents, emulsifying and suspending agents, sweetening, flavoring, and perfuming agents.

In some embodiments, injectable preparations, for example, sterile injectable aqueous or oleaginous suspensions are formulated according in any suitable manner, e.g., using dispersing agents, wetting agents and/or suspending agents. The sterile injectable preparation is, optionally, a sterile injectable solution, suspension, or emulsion in a nontoxic parenterally acceptable diluent or solvent, for example, as a solution in 1,3-butanediol. Among the acceptable vehicles and solvents that are optionally employed are water, Ringer's solution, U.S.P. and isotonic sodium chloride solution. In addition, sterile, fixed oils are conventionally employed as a solvent or suspending medium. For this purpose any bland fixed oil is optionally employed including synthetic mono- or diglycerides. In additional embodiments, fatty acids such as oleic acid are used in the preparation of injectables. In a specific embodiment, the polymer bioconjugate is solubilized in a carrier fluid comprising 1% (w/v) sodium carboxymethyl cellulose and 0.1% (v/v) Tween 80.

In some embodiments, the injectable formulations are sterilized, for example, by filtration through a bacteria-retaining filter, or by incorporating sterilizing agents in the form of sterile solid compositions which are optionally dissolved or dispersed in sterile water or other sterile injectable medium prior to use.

In certain embodiments, compositions for rectal or vaginal administration are suppositories. Suppositories are optionally prepared by mixing the therapeutic agent comprising polymer bioconjugates provided herein with suitable non-irritating excipients or carriers such as cocoa butter, polyethylene glycol, or a suppository wax which are solid at ambient temperature but liquid at body temperature and therefore melt in the rectum or vaginal cavity and release the therapeutic agent comprising polymer bioconjugates provided herein. As used herein, a “therapeutic agent comprising polymer bioconjugates provided herein” is used interchangeable with one or more polymer bioconjugate provided herein comprising a one or more RNAi therapeutic agent.

Suitable solid dosage forms for oral administration include, by way of non-limiting example, capsules, tablets, pills, powders, and granules. In such solid dosage forms, the polymer bioconjugates comprising an RNAi therapeutic agent (e.g., oligonucleotide) are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type are also optionally employed as fillers in soft and hard-filled gelatin capsules using such excipients as lactose or milk sugar as well as high molecular weight polyethylene glycols and the like.

In some embodiments, the solid dosage forms of tablets, dragees, capsules, pills, and granules are prepared with coatings and shells such as enteric coatings and other suitable coatings. They optionally contain opacifying agents. In certain embodiments, they are of a composition that they release the active ingredient(s) only, or preferentially, in a certain part of the intestinal tract, optionally, in a delayed manner. Examples of suitable embedding compositions include, by way of non-limiting example, polymeric substances and waxes.

Solid compositions of a similar type are optionally employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include, by way of non-limiting example, ointments, pastes, creams, lotions, gels, powders, solutions, sprays, inhalants, or patches. In some embodiments, therapeutic agents comprising polymer bioconjugates provided herein are admixed under sterile conditions with a pharmaceutically acceptable carrier and, optionally, one or more preservative, one or more buffer, or a combination thereof (e.g., as may be required). Ophthalmic formulation, ear drops, and eye drops are also contemplated as being within the scope of this invention.

Ointments, pastes, creams, and gels provided herein optionally contain, in addition to the therapeutic agent comprising polymer bioconjugates provided herein, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays optionally contain, in addition to therapeutic agent comprising polymer bioconjugates provided herein, excipients such as lactose, talc, silicic acid, aluminum hydroxide, calcium silicates, and polyimide powder, or mixtures of these substances. In some embodiments, sprays additionally contain customary propellants such as chlorofluorohydrocarbons.

Transdermal patches have the added advantage of providing controlled delivery of a compound to the body. Such dosage forms are made in any suitable manner, e.g., by dissolving or dispensing the microparticles or nanoparticles in a proper medium. Absorption enhancers are optionally used to increase the flux of the compound across the skin. The rate can be controlled by either providing a rate controlling membrane or by dispersing therapeutic agent comprising polymer bioconjugates provided herein in a polymer matrix or gel.

In some aspects of the invention, the polymer bioconjugates provide some properties (e.g. mechanical, thermal, etc.) that are usually performed by excipients, thus decreasing the amount of such excipients required for the formulation.

### EXAMPLES

Throughout the description of the present invention, various known acronyms and abbreviations are used to describe monomers or monomeric residues derived from polymerization of such monomers. Without limitation, unless otherwise noted: “BMA” (or the letter “B” as equivalent shorthand notation) represents butyl methacrylate or monomeric residue derived therefrom; “DMAEMA” (or the letter “D” as equivalent shorthand notation) represents N,N-dimethylaminoethyl methacrylate or monomeric residue derived therefrom; “Gal” refers to galactose or a galactose residue, optionally including hydroxyl-protecting moieties (e.g., acetyl) or to a pegylated derivative thereof (as described below); HPMA represents 2-hydroxypropyl methacrylate or monomeric residue derived therefrom; “MAA” represents methylacrylic acid or monomeric residue derived therefrom; “MAA(NHS)” represents N-hydroxyl-succinimide ester of methacrylic acid or monomeric residue derived therefrom; “PAA” (or the letter “P” as equivalent shorthand notation) represents 2-propylacrylic acid or monomeric residue derived therefrom, “PEGMA” refers to the pegylated methacrylic monomer, CH₃—O—(CH₂O)₇₋₈OC(O)C(CH₃)CH₂ or monomeric residue derived therefrom. In each case, any such designation indicates the monomer (including all salts, or ionic analogs thereof), or a monomeric residue derived from polymerization of the monomer (including all salts or ionic analogs thereof), and the specific indicated form is evident by context to a person of skill in the art.

### Example 1

### Synthesis of Unimeric, Single Block Copolymers Using Raft Polymerization

### A. RAFT Chain Transfer Agent, ECT.

The synthesis of the chain transfer agent (CTA), 4-Cyano-4-(ethylsulfanylthiocarbonyl) sulfanylpentanoic acid (ECT), utilized for some RAFT polymerizations, is adapted from a procedure by Moad et al., Polymer, 2005, 46(19): 8458-68. Briefly, ethane thiol (4.72 g, 76 mmol) is added over 10 minutes to a stirred suspension of sodium hydride (60% in oil) (3.15 g, 79 mmol) in diethyl ether (150 ml) at 0° C. The solution is then allowed to stir for 10 minutes prior to the addition of carbon disulfide (6.0 g, 79 mmol). Crude sodium S-ethyl trithiocarbonate (7.85 g, 0.049 mol) is collected by filtration, suspended in diethyl ether (100 mL), and reacted with Iodine (6.3 g, 0.025 mol). After 1 hour the solution is filtered, washed with aqueous sodium thiosulfate, and dried over sodium sulfate. The crude bis(ethylsulfanylthiocarbonyl) disulfide is then isolated by rotary evaporation. A solution of bis-(ethylsulfanylthiocarbonyl) disulfide (137 g, 0.005 mol) and 4,4′-azobis(4-cyanopentanoic acid) (2.10 g, 0.0075 mol) in ethyl acetate (50 mL) is heated at reflux for 18 h. Following rotary evaporation of the solvent, the crude 4-Cyano-4 (ethylsulfanylthiocarbonyl) sulfanylvpentanoic acid (ECT) is isolated by column chromatography using silica gel as the stationary phase and 50:50 ethyl acetate hexane as the eluent.

### B. Block Copolymerization of PAA, BA or PAA, BA, AA-NHS.

[PAAₓ-BA_{y}]ₘ
[PAAₓ-BA_{y}-AA(NHS)_{z}]ₘ

RAFT polymerization is conducted in DMF at 70° C. under a nitrogen atmosphere for 6-18 hours using ECT and 2,2′-Azobis(4-methoxy-2.4-dimethyl valeronitrile) (V-70) (Wako chemicals) as the radical initiator. The desired stoichiometric quantities of PAA (propylacrylic acid) and BA (butyl acrylate) or PAA, BA, and AA-NHS (acrylic acid N-hydroxysuccinimide ester) are dissolved in N,N-dimethylformamide. For all polymerizations [M]ₒ/[CTA]ₒ and [CTA]ₒ/[I]ₒ are 150:1 and 10:1 respectively. Following the addition of V70 the solutions are purged with nitrogen for 30 min and allowed to react at 70° C. for 6-18 h. The resultant diblock copolymers are isolated by precipitation into 50:50 v:v diethyl ether/pentane. The precipitated polymers are then redissolved in acetone and subsequently precipitated into pentane (x3) and dried overnight in vacuo. Gel permeation chromatography (GPC) is used to determine molecular weights and polydispersities (PDI, M_{w}/Mₙ) of the diblock copolymer samples in DMF with respect to polymethyl methacrylate standards (SEC Tosoh TSK-GEL R-3000 and R-4000 columns (Tosoh Bioscience, Montgomeryville, Pa.) connected in series to a Viscotek GPCmax VE2001 and refractometer VE3580 (Viscotek, Houston, Tex.). HPLC-grade DMF containing 1.0 wt % LiBr is used as the mobile phase. The desired molecular weight of the di-block copolymers (typically between 10,000 and 30,000 Da) is determined by kinetic analysis where reaction times are varied between 6-18 hrs.

### Example 2

### Synthesis of Unimeric, Single Block, Ligand-Targeting Copolymers

[PAAₓ-BA_{y}-AA(L)_{z}]ₘ

L=galactose, lactose, or any amine containing ligand, peptide, or protein

A copolymer of PAA, BA, AA-NHS is synthesized as described in example 1. The approximate desired composition is obtained by selecting the corresponding molar ratios of PAA, BA, AA-NHS in the polymerization reaction mixture, for example, 40% PAA, 40% BA and 20% AA-NHS. Following the above described purification, the polymer is dissolved in ethanol at 2-20 mg/mL and diluted 10-fold in 0.1 M phosphate buffer, pH 7.4. Small molecule targeting ligands with amino groups (for example, galactosamine, lactosamine) were added in 2-5-fold molar excess to NHS groups and reacted for 1-6 hours at room temperature to form the ligand amide derivative. The extent of reaction is determined by measuring the increase in absorptivity at 260 nm of the NHS group upon cleavage using a molar extinction coefficient of 8.2×103 M⁻¹cm⁻¹ (pH 9). Following the reaction, the ligand modified polymer is extensively dialysed to remove unreacted ligand and lyophilized.

### Example 3

### Synthesis of Unimeric, Single Block Copolymers Conjugated to siRNA

Pyridyl disulfide functionalized chain transfer agent (CTA), 3-(pyridin-2-ylsulfanyl)-2-(ethylthiocarbonothioylthio) propanoate.

3-(2-pyridinyldisulfanyl)propanol and 2-(ethyl thiocarbonate)propionic acid are synthesized as previously described (Murthy et al, Bioconjugate Chemistry 2003, 14, 412-419; Wood et al., Organic letters 2006, 8, 553-556). 3-(2-pyridinyldisulfanyl)propanol (124.3 mg, 0.618 mmol) and 2-(ethyl thiocarbonate)propionic acid (137 mg, 0.680 mmol) were dissolved in dichloromethane (25 mL) and cooled to 0° C. 1-Ethyl-3-(3-dimethylaminopropyl carbodiimide hydrochloride (130.7 mg, 0.680 mmol) and 4-dimethylaminopyridine (7.8 mg, 0.0618 mmol) were added and the reaction mixture was warmed to room temperature with constant stirring. After 12 hrs, the crude product was concentrated and purified by silica gel chromatography (2:1 hexane:ethyl acetate yielding pyridyl disulfide CTA as a yellow oil.

Pyridyl Disulfide Functionalized Polymer (α-End)
Pyr-S—S—[PAAₓ-BA_{y}-AA(NHS)A_{z}]ₘ

Polymerization of PAA, BA, AA-NHS with a pyridyl disulfide moiety at the α-end of the polymer is conducted as described in Example 1B, except that pyridyl disulfide functionalized CTA is used as the chain transfer agent in place of ECT and the reaction temperature is 60° C.

Conjugation of siRNA to pyridyl disulfide end functionalized poly[PAA-BA-AA(NHS)].
siRNA-S—S-[PAAₓ-BA_{y}-AA(NHS)_{z}]ₘ

siRNA containing a disulfide attached by a 6-carbon linker to the 5′ of the sense strand (Agilent) was reduced to the free thiol by reaction with 100 mM dithiothreitol at room temperature for 3 hours, followed by ethanol precipitation to remove unreacted DTT. The siRNA precipitate is resuspended in 400 ul of 0.1 M sodium bicarbonate buffer pH 8.5 containing pyridyl disulfide end functionalized poly[PAA-BA-AA(NHS)] at a molar ratio of siRNA to polymer of 2:1. The disulfide exchange reaction is allowed to proceed for 8-12 hours at room temperature. The siRNA-polymer conjugate is purified by gel filtration chromatography.

Conjugation of targeting ligand to siRNA-poly[PAA-BA-AA(NHS)]
siRNA-S—S-[PAAₓ-BA_{y}-AA(L)_{z}]ₘ

siRNA-polymer conjugate is dialyzed against 0.1 M phosphate buffer, pH 7.4. A targeting ligand containing a free amino group (for example, galactosamine or peptide) is added in 2-5-fold molar excess to polymer NHS groups and reacted for 1-6 hours at room temperature to form the ligand amide derivative, followed by dialysis against phosphate buffer or gel filtration chromatography to remove unreacted ligand.

### Example 4

### Functional Design of Poly[HPMN]-b-[(PAA)ₓ(BMA)_{y}(DMAEMA)_{z}]ₘ

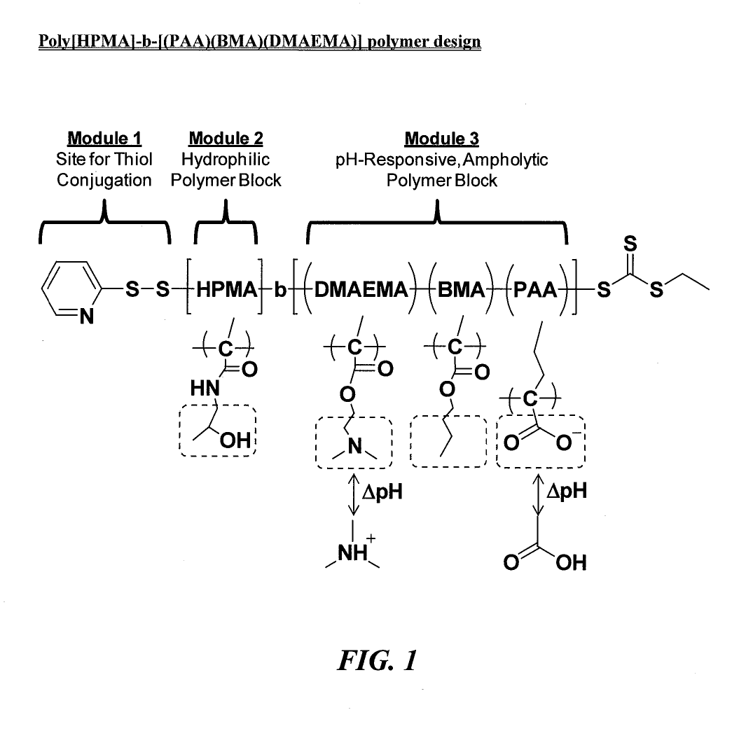

FIG. 1 shows the polymer design for Poly[HPMA]-b-[(PAA)ₓ(BMA)_{y}(DMAEMA)_{z}]ₘ. Multifunctional properties were incorporated via RAFT polymer synthesis strategies using a pyridyl disulfide end-functionalized CTA to form a diblock architecture designed to possess aqueous solubility and pH-dependent membrane destabilizing properties. The monomer chemical functionalities highlighted in FIG. 1 were chosen in order to produce the desired properties for each polymer block. Module 3 was designed to be near charge neutrality at physiologic pH (approximately 50% DMAEMA protonation and 50% PAA deprotonation predicted) and to undergo a transition to a more hydrophobic and positively charged state in lower pH environments.

### Example 5

### Preparation of Thiol Reactive Polymer

Synthesis of Trithiocarbonic acid 1-cyano-1-methyl-3-[2-(pyridin-2-yldisulfanyl)-ethylcarbamoyl]-propyl ester ethyl ester (PyrECT).

The 4-Cyano-4-(ethylsulfanylthiocarbonyl) sulfanylvpentanoic acid (ECT) precursor was synthesized as Shown in Scheme 1. The pyridyl disulfide functionalized RAFT chain transfer agent (CTA) was synthesized by first converting ECT to the NHS ester followed by reaction with Pyridyldithio-ethylamine.

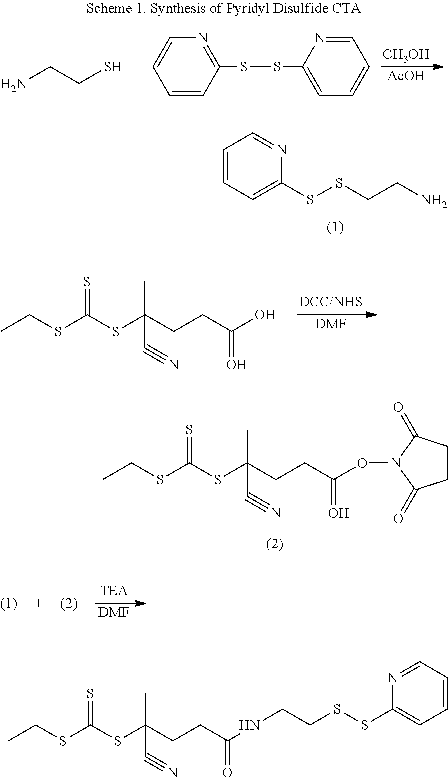

ECT (1.05 g, 4 mmol) and N-hydroxysuccinimide (0.460 g, 4 mmol) were dissolved in 100 mL of chloroform. The mixture was then cooled to 0° C. at which time N,N′ dicyclohexylcarbodiimide (0.865 mg, 4.2 mmol) was added. The solution was maintained at 0° C. for 1 hour and then allowed to react at room temperature for 22 hours. The solution was then filtered to remove the dicyclohexyl urea and the solution concentrated via rotary evaporation. The resultant solid was then dried under vacuum and used without any further purification. NHS ECT (1.80 g, 5.0 mmol) and pyridyldithio-ethylamine (0.90 g, 5.0 mmol.) where then separately dissolved in 200 and 300 mL of chloroform, respectively. The solution of pyridyldithio-ethylamine was then added dropwise as three fractions 20 minutes apart. The mixture was then allowed to react at room temperature for 2 hours. After solvent removal, two successive column chromatographies (Silica gel 60, Merk) were performed (ethyl Acetate:hexane 50:50; ethyl acetate:hexane 70:30 v/v) yielding a viscous orange solid. ¹H NMR 200 MHz (CDCl₃, RT, ppm) 1.29-1.41 [t, CH3CH2S: 3H], 1.85-1.93 [s, (CH₃)C(CN): 3H], 2.33-2.59 [m, C(CH₃)(CN)(CH₂CH₂): 4H], 2.86-2.97 [t, CH₂SS: 2H], 3.50-3.61 [t, NHCH₂: 2H], 7.11-7.22 [m, Ar Para CH: 1H], 7.46-7.52 [m, Ar CH Ortho: 1H], 7.53-7.62 [br, NH: 1H], 7.53-7.68 [m, Ar meta CH: 1H], 8.47-8.60 [m, meta CHN, 1H].

### Example 6

RAFT Polymerization of Pyridyl Disulfide Functionalized Poly[HPMA]-b-[(PAA)ₓ(BMA)_{y}(DMAEMA)_{z}]ₘ.

The RAFT polymerization of N-(2-hydroxypropyl)methacrylamide (HPMA) was conducted in methanol (50 weight percent monomer:solvent) at 70° C. under a nitrogen atmosphere for 8 hours using 2,2′-azo-bis-isobutyrylnitrile (AIBN) as the free radical initiator. The molar ratio of CTA to AIBN was 10 to 1 and the monomer to CTA ratio was set so that a molecular weight of 25,000 g/mol would be achieved if at 100% conversion. The poly(HPMA) macro-CTA was isolated by repeated precipitation into diethyl ether from methanol.

The macro-CTA was dried under vacuum for 24 hours and then used for block copolymerization of dimethylaminoethyl methacrylate (DMAEMA), propylacrylic acid (PAA), and butyl methacrylate (BMA). Equimolar quantities of DMAEMA, PAA, and BMA ([M]ₒ/[CTA]ₒ=250) were added to the HPMA macroCTA dissolved in N,N-dimethylformamide (25 wt % monomer and macroCTA to solvent). The radical initiator V70 was added with a CTA to initiator ratio of 10 to 1. The polymerization was allowed to proceed under a nitrogen atmosphere for 18 hours at 30° C. Afterwards, the resultant diblock polymer was isolated by precipitation 4 times into 50:50 diethyl ether/pentane, redissolving in ethanol between precipitations. The product was then washed 1 time with diethyl ether and dried overnight in vacuo.

Gel permeation chromatography (GPC) was used to determine molecular weight and polydispersity (M_{w}/Mₙ, PDI) of both the poly(HPMA) macroCTA and the diblock copolymer in DMF. Molecular weight calculations were based on column elution times relative to polymethyl methacrylate standards using HPLC-grade DMF containing 0.1 wt % LiBr at 60° C. as the mobile phase. Tris(2-carboxyethyl) phosphine hydrochloride (TCEP) was used to reduce the polymer end pyridyl disulfide, releasing 2-pyridinethione. Based on the experimentally determined polymer molecular weight and the molar extinction coefficient of 2-pyridinethione at 343 nm (8080 M^{-l}cm⁻¹) in aqueous solvents, percent end group preservation was determined for the poly(HPMA) macroCTA and the diblock copolymer.

### Example 7

### Polymer-siRNA Conjugation

siRNA Conjugation to HPMA-PDSMA Co-Polymer:

Thiolated siRNA was obtained commercially (Agilent, Boulder, Colo.) as a duplex RNA with a disulfide modified 5′-sense strand. The free thiol form for conjugation is prepared by dissolving the lyophilized compound in water and treated for 1 hour with the disulfide reducing agent TCEP immobilized within an agarose gel. The reduced RNA (400 nM) is then reacted for 24 hours with the pyridyl disulfide-functionalized polymer in phosphate buffer (pH 7) containing 5 mM ethylenediaminetetraacetic acid (EDTA).

The reaction of the pyridyl disulfide polymer with the RNA thiol creates 2-pyridinethione, which is spectrophotometrically measured to characterize conjugation efficiency. To further validate disulfide exchange, the conjugates are run on an SDS-PAGE 16.5% tricine gel. In parallel, aliquots of the conjugation reactions are treated with immobilized TCEP prior to SDS-PAGE to verify release of the RNA from the polymer in a reducing environment. Conjugation reactions are conducted at polymer/RNA stoichiometries of 1, 2, and 5. UV spectrophotometric absorbance measurements at 343 nm for 2-pyridinethione release are used to measure conjugation efficiencies.

### Example 8

### Polymer Conjugation to a Pyridiyl Disulfide Functionalized Biomolecular Agent

Conjugation to a biomolecular agent (e.g., an antibody, siRNA or the like) is carried out using a cell-internalized form of the biomolecular agent containing a carboxy-terminal cysteine residue. This thiol-containing amino acid is utilized to react with a pyridyl disulfide functionalized polymer as shown in Scheme 2 below. Spectrophotometric measurement of the reaction byproduct 2-pyridinethione (, ε=8080 M⁻¹cm⁻¹, 343 nm) and SDS-PAGE gels are utilized to confirm peptide-polymer conjugation.

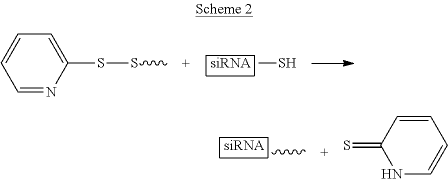

1-61. (canceled)

62. A polymer bioconjugate, comprising:
(a) a pH-dependent membrane-destabilizing polymer having an alpha end and an omega end, and comprising:
(i) a plurality of chargeable monomeric units comprising a species that is anionic at about neutral pH; and
(ii) a plurality of monomeric units comprising a hydrophobic group; and
(b) an RNAi agent covalently coupled to the alpha end or the omega end of the membrane-destabilizing polymer,
provided that the polymer bioconjugate does not self-assemble into a micelle when the polymer bioconjugate is at or below a concentration of 100 μg/mL in water at a neutral pH.

63. The polymer bioconjugate of claim 62, wherein the pH-dependent membrane-destabilizing polymer is a block copolymer.

64. The polymer bioconjugate of claim 62, wherein the polymer bioconjugate has a particle size diameter of not more than 30 nm.

65. The polymer bioconjugate of claim 62, wherein the RNAi agent is a polynucleotide.

66. The polymer bioconjugate of claim 65, wherein the polynucleotide is a dicer substrate.

67. The polymer bioconjugate of claim 62, wherein the RNAi agent is covalently coupled to the membrane-destabilizing polymer through a linking moiety.

68. The polymer bioconjugate of claim 67, wherein the linking moiety is cleavable and comprises a dicer substrate or a disulfide.

69. The polymer bioconjugate of claim 62, wherein the species that are anionic at about neutral pH are non-charged or substantially neutral at an acidic pH.

70. The polymer bioconjugate of claim 62 further comprising one or more targeting ligands covalently coupled to the pH-dependent membrane-destabilizing polymer.

71. The polymer bioconjugate of claim 70, wherein the one or more targeting ligands are selected from the group consisting of a vitamin, a saccharide, a peptide, a hormone, an aptamer, a small molecule, or combinations thereof.

72. The polymer bioconjugate of claim 71, wherein the targeting ligand is covalently coupled to the end of the membrane-destabilizing polymer opposing the RNAi agent.

73. The polymer bioconjugate of claim 62, wherein the membrane-destabilizing polymer comprises x monomeric units comprising the species that are anionic at about neutral pH and y hydrophobic monomeric units comprising the hydrophobic groups, wherein the ratio of x to y is about 1:1 to about 5:1.

74. The polymer bioconjugate of claim 73, wherein at least one of the monomeric units comprises alkyl acrylic acid as the species that is anionic at about neutral pH.

75. A compound comprising a plurality of polymer bioconjugates of claim 71, each polymer bioconjugate comprising a membrane-destabilizing polymer coupled to the same targeting ligand, and coupled to the RNAi agent.

76. The compound of claim 75, wherein each membrane-destabilizing polymer is coupled to the RNAi agent at either the alpha or omega end of the polymer, and is coupled to the targeting ligand at the opposing end of the polymer.

77. A compound, comprising:
a polymer bioconjugate comprising
(a) a pH-dependent membrane-destabilizing polymer having an alpha end and an omega end, and comprising
(i) a plurality of monomeric units comprising a species that is anionic at about neutral pH; and
(ii) a plurality of monomeric units comprising hydrophobic groups, and
(b) an RNAi agent covalently coupled to the alpha end or the omega end of the membrane-destabilizing polymer; and
(c) a proteinaceous targeting ligand covalently coupled to the pH-dependent membrane-destabilizing polymer.

78. The compound of claim 77, comprising a plurality of polymer bioconjugates covalently coupled to the proteinaceous targeting ligand.

79. The compound of claim 78, comprising at least 2 polymer bioconjugates covalently coupled to the proteinaceous targeting ligand.

80. The compound of claim 79, wherein the proteinaceous targeting ligand is covalently coupled to the pH dependent membrane destabilizing polymer at the end opposite the RNAi agent.

81. The compound of claim 79, wherein the proteinaceous targeting ligand is covalently coupled to the pH dependent membrane destabilizing polymer at a pendant group of a monomeric unit of the pH dependent membrane destabilizing polymer.

82. A composition comprising the polymer bioconjugate of claim 62.

83. A method for intracellular delivery of an RNAi agent, comprising contacting a cell with the polymer bioconjugate of claim 62.
